SEQUENCE LISTING (97 sequences)

SEQ ID NO: 1 - protein, 7 aa, Artificial Sequence
  MPER sequence
KWPWIWL

SEQ ID NO: 2 - protein, 4 aa, Artificial Sequence
  AQGV-peptide
AQGV

SEQ ID NO: 3 - protein, 4 aa, Artificial Sequence
  LQGV-peptide
LQGV

SEQ ID NO: 4 - protein, 4 aa, Artificial Sequence
  AQGV analogue
AQLP

SEQ ID NO: 5 - protein, 4 aa, Artificial Sequence
  AQGV analogue
PLQA

SEQ ID NO: 6 - protein, 4 aa, Artificial Sequence
  AQGV analogue
LQGV

SEQ ID NO: 7 - protein, 4 aa, Artificial Sequence
  AQGV analogue
LAGV

SEQ ID NO: 8 - protein, 4 aa, Artificial Sequence
  AQGV analogue
PQVG

SEQ ID NO: 9 - protein, 4 aa, Artificial Sequence
  AQGV analogue
PQVA

SEQ ID NO: 10 - protein, 4 aa, Artificial Sequence
  AQGV analogue
PQVR

SEQ ID NO: 11 - protein, 4 aa, Artificial Sequence
  AQGV analogue
VGQL

SEQ ID NO: 12 - protein, 4 aa, Artificial Sequence
  AQGV analogue
LQPL

SEQ ID NO: 13 - protein, 4 aa, Artificial Sequence
  AQGV analogue
RQGV

SEQ ID NO: 14 - protein, 4 aa, Artificial Sequence
  AQGV analogue
LQVG

SEQ ID NO: 15 - protein, 4 aa, Artificial Sequence
  AQGV analogue
LQGA

SEQ ID NO: 16 - protein, 4 aa, Artificial Sequence
  AQGV analogue
LQGR

SEQ ID NO: 17 - protein, 4 aa, Artificial Sequence
  AQGV analogue
AQGA

SEQ ID NO: 18 - protein, 4 aa, Artificial Sequence
  AQGV analogue
QPLA

SEQ ID NO: 19 - protein, 4 aa, Artificial Sequence
  AQGV analogue
PQVP

SEQ ID NO: 20 - protein, 4 aa, Artificial Sequence
  AQGV analogue
VGQA

SEQ ID NO: 21 - protein, 4 aa, Artificial Sequence
  AQGV analogue
QVGQ

SEQ ID NO: 22 - protein, 4 aa, Artificial Sequence
  AQGV analogue
VGQG

SEQ ID NO: 23 - protein, 4 aa, Artificial Sequence
  peptide
AGQV

SEQ ID NO: 24 - protein, 4 aa, Artificial Sequence
  tetrapeptide
QQGV

SEQ ID NO: 25 - protein, 4 aa, Artificial Sequence
  tetrapeptide
LQGV

SEQ ID NO: 26 - protein, 4 aa, Artificial Sequence
  tetrapeptide
GQGV

SEQ ID NO: 27 - protein, 4 aa, Artificial Sequence
  tetrapeptide
PQGV

SEQ ID NO: 28 - protein, 4 aa, Artificial Sequence
  tetrapeptide
VQGV

SEQ ID NO: 29 - protein, 4 aa, Artificial Sequence
  tetrapeptide
ALGV

SEQ ID NO: 30 - protein, 4 aa, Artificial Sequence
  tetrapeptide
LLGV

SEQ ID NO: 31 - protein, 4 aa, Artificial Sequence
  tetrapeptide
QLGV

SEQ ID NO: 32 - protein, 4 aa, Artificial Sequence
  tetrapeptide
GLGV

SEQ ID NO: 33 - protein, 4 aa, Artificial Sequence
  tetrapeptide
PLGV

SEQ ID NO: 34 - protein, 4 aa, Artificial Sequence
  tetrapeptide
VLGV

SEQ ID NO: 35 - protein, 4 aa, Artificial Sequence
  tetrapeptide
QLGV

SEQ ID NO: 36 - protein, 4 aa, Artificial Sequence
  tetrapeptide
QAGV

SEQ ID NO: 37 - protein, 4 aa, Artificial Sequence
  tetrapeptide
QGGV

SEQ ID NO: 38 - protein, 4 aa, Artificial Sequence
  tetrapeptide
QPGV

SEQ ID NO: 39 - protein, 4 aa, Artificial Sequence
  tetrapeptide
QVGV

SEQ ID NO: 40 - protein, 4 aa, Artificial Sequence
  tetrapeptide
LAGV

SEQ ID NO: 41 - protein, 4 aa, Artificial Sequence
  tetrapeptide
LGGV

SEQ ID NO: 42 - protein, 4 aa, Artificial Sequence
  tetrapeptide
LPGV

SEQ ID NO: 43 - protein, 4 aa, Artificial Sequence
  tetrapeptide
LVGV

SEQ ID NO: 44 - protein, 8 aa, Artificial Sequence
  SARS-COV-2 spike protein peptide-motif sequence
KWPWYIWL

SEQ ID NO: 45 - protein, 8 aa, Artificial Sequence
  SARS-COV-2 spike protein peptide-motif sequence variant
KWPWYVWL

SEQ ID NO: 46 - protein, 18 aa, Artificial Sequence
  MPER peptide
LGKYEQYIKWPWYVWLGF

SEQ ID NO: 47 - protein, 18 aa, Artificial Sequence
  MPER peptide
KWPWYVWLGFIAGLIAIV

SEQ ID NO: 48 - protein, 8 aa, Artificial Sequence
  peptide
  MISC_FEATURE (8)..(8): linked to maleate
AQGVLPGQ

SEQ ID NO: 49 - protein, 8 aa, Artificial Sequence
  peptide
  MISC_FEATURE (8)..(8): Linked to maleate
LQGVLPGQ

SEQ ID NO: 50 - protein, 8 aa, Artificial Sequence
  peptide
  MISC_FEATURE (8)..(8): linked to maleate
AQGLQPGQ

SEQ ID NO: 51 - protein, 8 aa, Artificial Sequence
  peptide
  MISC_FEATURE (8)..(8): linked to maleate
LQGLQPGQ

SEQ ID NO: 52 - protein, 4 aa, Artificial Sequence
  peptide
  MISC_FEATURE (4)..(4): linked to maleate
AQGV

SEQ ID NO: 53 - protein, 5 aa, Artificial Sequence
  peptide
  MISC_FEATURE (5)..(5): linked to maleate
LQGVL

SEQ ID NO: 54 - protein, 5 aa, Artificial Sequence
  peptide
  MISC_FEATURE (5)..(5): linked to maleate
AQGLQ

SEQ ID NO: 55 - protein, 5 aa, Artificial Sequence
  peptide
  MISC_FEATURE (5)..(5): linked to maleate
LQGLQ

SEQ ID NO: 56 - protein, 8 aa, Artificial Sequence
  peptide
  MISC_FEATURE (8)..(8): linked to acetate
AQGVLPGQ

SEQ ID NO: 57 - protein, 8 aa, Artificial Sequence
  peptide
  MISC_FEATURE (8)..(8): linked to acetate
LQGVLPGQ

SEQ ID NO: 58 - protein, 8 aa, Artificial Sequence
  peptide
  MISC_FEATURE (8)..(8): linked to acetate
AQGLQPGQ

SEQ ID NO: 59 - protein, 8 aa, Artificial Sequence
  peptide
  MISC_FEATURE (8)..(8): linked to acetate
LQGLQPGQ

SEQ ID NO: 60 - protein, 4 aa, Artificial Sequence
  peptide
  MISC_FEATURE (4)..(4): linked to acetate
AQGV

SEQ ID NO: 61 - protein, 5 aa, Artificial Sequence
  peptide
  MISC_FEATURE (5)..(5): linked to acetate
LQGVL

SEQ ID NO: 62 - protein, 5 aa, Artificial Sequence
  peptide
  MISC_FEATURE (5)..(5): linked to acetate
AQGLQ

SEQ ID NO: 63 - protein, 5 aa, Artificial Sequence
  peptide
  MISC_FEATURE (5)..(5): linked to acetate
LQGLQ

SEQ ID NO: 64 - protein, 8 aa, Artificial Sequence
  peptide
  MISC_FEATURE (8)..(8): linked to tartrate
AQGVLPGQ

SEQ ID NO: 65 - protein, 8 aa, Artificial Sequence
  peptide
  MISC_FEATURE (8)..(8): linked to tartrate
LQGVLPGQ

SEQ ID NO: 66 - protein, 8 aa, Artificial Sequence
  peptide
  MISC_FEATURE (8)..(8): linked to tartrate
AQGLQPGQ

SEQ ID NO: 67 - protein, 8 aa, Artificial Sequence
  peptide
  MISC_FEATURE (8)..(8): linked to tartrate
LQGLQPGQ

SEQ ID NO: 68 - protein, 4 aa, Artificial Sequence
  peptide
  MISC_FEATURE (4)..(4): linked to tartrate
AQGV

SEQ ID NO: 69 - protein, 5 aa, Artificial Sequence
  peptide
  MISC_FEATURE (5)..(5): linked to tartrate
LQGVL

SEQ ID NO: 70 - protein, 5 aa, Artificial Sequence
  peptide
  MISC_FEATURE (5)..(5): linked to tartrate
AQGLQ

SEQ ID NO: 71 - protein, 5 aa, Artificial Sequence
  peptide
  MISC_FEATURE (5)..(5): linked to tartrate
LQGLQ

SEQ ID NO: 72 - protein, 8 aa, Artificial Sequence
  peptide
  MISC_FEATURE (8)..(8): linked to citrate
AQGVLPGQ

SEQ ID NO: 73 - protein, 8 aa, Artificial Sequence
  peptide
  MISC_FEATURE (8)..(8): linked to citrate
LQGVLPGQ

SEQ ID NO: 74 - protein, 8 aa, Artificial Sequence
  peptide
  MISC_FEATURE (8)..(8): linked to citrate
AQGLQPGQ

SEQ ID NO: 75 - protein, 8 aa, Artificial Sequence
  peptide
  MISC_FEATURE (8)..(8): linked to citrate
LQGLQPGQ

SEQ ID NO: 76 - protein, 4 aa, Artificial Sequence
  peptide
  MISC_FEATURE (4)..(4): linked to citrate
AQGV

SEQ ID NO: 77 - protein, 5 aa, Artificial Sequence
  peptide
  MISC_FEATURE (5)..(5): linked to citrate
LQGVL

SEQ ID NO: 78 - protein, 5 aa, Artificial Sequence
  peptide
  MISC_FEATURE (5)..(5): linked to citrate
AQGLQ

SEQ ID NO: 79 - protein, 5 aa, Artificial Sequence
  peptide
  MISC_FEATURE (5)..(5): linked to citrate
LQGLQ

SEQ ID NO: 80 - protein, 49 aa, Artificial Sequence
  CoV-229E spike protein
LQTLIDNINSTLVDLKWLNRVETYIKWPWWVWLCISVVLIFVVSMLLLC

SEQ ID NO: 81 - protein, 49 aa, Artificial Sequence
  CoV-NL63 spike protein
LQGLIDQINSTYVDLKLLNRFENYIKWPWWVWLIISVVFVVLLSLLVFC

SEQ ID NO: 82 - protein, 49 aa, Artificial Sequence
  CoV-HKU1 spike protein
IQESIKSLNSSFINLKEIGTYEMYVKWPWYIWLLIVILFIIFLMILFFI

SEQ ID NO: 83 - protein, 49 aa, Artificial Sequence
  CoV-OC43 spike protein
LQEAIKVLNQSYINLKDIGTYEYYVKWPWYVWLLICLAGVAMLVLLFFI

SEQ ID NO: 84 - protein, 49 aa, Artificial Sequence
  MERS spike protein
LQQVVKALNESYIDLKELGNYTYYNKWPWYIWLGFIAGLVALALCVFFI

SEQ ID NO: 85 - protein, 49 aa, Artificial Sequence
  SARS-CoV spike protein
LNEVAKNLNESLIDLQELGKYEQYIKWPWYVWLGFIAGLIAIVMVTILL

SEQ ID NO: 86 - protein, 49 aa, Artificial Sequence
  SARS CoV-2 spike protein
LNEVAKNLNESLIDLQELGKYEQYIKWPWYIWLGFIAGLIAIVMVTIML

SEQ ID NO: 87 - protein, 18 aa, Artificial Sequence
  SARS-CoV-1 MPER peptide
LGKYEQYIKWPWYVWLGF

SEQ ID NO: 88 - protein, 18 aa, Artificial Sequence
  SARS-CoV-1 MPER peptide
KWPWYVWLGFIAGLIAIV

SEQ ID NO: 89 - protein, 10 aa, Artificial Sequence
  SARS-CoV-1 Fusogenic sequence
KWPWYVWLGF

SEQ ID NO: 90 - protein, 10 aa, Artificial Sequence
  SARS-CoV-2 Fusogenic sequence
KWPWYIWLGF

SEQ ID NO: 91 - protein, 10 aa, Artificial Sequence
  MERS-CoV Fusogenic sequence
KWPWYIWLGF

SEQ ID NO: 92 - protein, 36 aa, Artificial Sequence
  part of CoV-OC43 spike protein
MNRLQEAIKVLNQSYINLKDIGTYEYYVKWPWYVWL

SEQ ID NO: 93 - protein, 36 aa, Artificial Sequence
  part of CoV-HKU-1 spike protein
MNLIQESIKSLNNSYINLKDIGTYEMYVKWPWYVWL

SEQ ID NO: 94 - protein, 36 aa, Artificial Sequence
  part of CoV-229E spike protein
VQKLQTLIDNINSTLVDLKWLNRVETYIKWPWWVWL

SEQ ID NO: 95 - protein, 36 aa, Artificial Sequence
  part of CoV-NL63 spike protein
TVELQGLIDQINSTYVDLKLLNRFENYIKWPWWVWL

SEQ ID NO: 96 - protein, 36 aa, Artificial Sequence
  part of SARS-CoV spike protein
IDRLNEVAKNLNESLIDLQELGKYEQYIKWPWYVWL

SEQ ID NO: 97 - protein, 36 aa, Artificial Sequence
  part of SARS-CoV-2 spike protein
IDRLNEVAKNLNESLIDLQELGKYEQYIKWPWYIWL

(19) United States
(12) Patent Application Publication
(10) Pub. No.: US 2023/0241155 A1
(43) Pub. Date: Aug. 3, 2023

(54) METHODS AND MEANS FOR MODIFYING HEMODYNAMICS IN INFECTIONS

(71) Applicant: Biotempt B.V., Den Haag (NL)

(72) Inventors: Gert Wensvoort, Koekange (NL); Eric Claassen, Lelystad (NL); Johan Renes, Amsterdam (NL)

(21) Appl. No.: 17/995,692

(22) PCT Filed: Apr. 6, 2021
(86) PCT No.: PCT/NL2021/050223

§ 371 Date: Oct. 6, 2022

Related U.S. Application Data
(60) Provisional application No. 63/005,999, filed on Apr. 6, 2020.
(60) Provisional application No. 63/045,737, filed on Jun. 29, 2020.
(60) Provisional application No. 63/085,771, filed on Sep. 30, 2020.

(51) Int. Cl. A61K 38/07; A61K 38/08; A61P 31/14; A61P 9/14

(52) U.S. Cl. CPC A61K 38/07; A61K 38/08; A61P 31/14; A61P 9/14

(57) ABSTRACT

Provided is a method of treatment comprising administering an AQGV peptide, or a functional analogue thereof, to a human subject, the human subject suffering from an infection, particularly a respiratory infection, more specifically, a viral infection, more in particular, a Corona virus infection, wherein the treatment of administering an AQGV peptide comprises maintaining or improving hemodynamic stability in the human subject.

### CROSS-REFERENCE TO RELATED APPLICATIONS

This application is a national phase entry under 35 U.S.C. § 371 of International Patent Application PCT/NL2021/050223, filed Apr. 6, 2021, designating the United States of America and published as International Patent Publication WO 2021/206547 A1 on Oct. 14, 2021, which claims the benefit under Article 8 of the Patent Cooperation Treaty to U.S. Provisional Patent Application Ser. No. 63/005,999, filed Apr. 6, 2020, to U.S. Provisional Patent Application Ser. No. 63/045,737, filed Jun. 29, 2020, and to U.S. Provisional Patent Application Ser. No. 63/085,771, filed Sep. 30, 2020.

### TECHNICAL FIELD

The application relates to methods and means for alleviating certain effects resulting from infection, in particular, hemodynamic effects. More specifically, the disclosure relates to peptide preparations used in the treatment of viral infections that affect the permeability of the vascular system.

### BACKGROUND

Recently, the world has been hit hard by a pandemic caused by a virus called SARS-Cov-2, a coronavirus causing COVID-19. The new coronavirus mainly seems to kill by flooding and clogging the tiny air sacs in the lungs with fluid, choking off the body's oxygen supply until it shuts down the organs essential for life. Such suffocation with one's own fluid seems a model of respiratory disease that more coronaviruses may be capable of inducing. A large reservoir of such viruses in various exotic animals may cause a similar pandemic with similar suffocation as SARS-Cov-2, considering our lack of pre-existing immunity. As virus-specific vaccines and/or antiviral agents will typically become available only after the infection has already spread throughout a large part of the population, other means and methods for treatment are dearly needed, as we may expect more of these types of viral infections, typically through zoonotic occurrences, where vaccines or anti-viral agents would not be specific enough or developed too late. Therefore there is a persisting and continuous need to at least be able to combat deleterious effects that these infections will have in common. The present disclosure provides means and methods to do just that.

### BRIEF SUMMARY

In a first embodiment, the disclosure provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, such as with a virus. In a preferred embodiment, the disclosure provides the method for reducing the gas diffusion distance between lung-alveoli and the vascular network surrounding alveoli in a human subject suffering from a respiratory infection, allowing reduction of fluid in alveoli and/or allowing improved oxygen supply to the subject's body. In a preferred embodiment, the substance comprises an AQGV-peptide (SEQ ID NO:2), an LQGV-peptide(SEQ ID NO:3), or a functional analogue of either. In a preferred embodiment, the disclosure provides the method for reducing the gas diffusion distance between lung-alveoli and the vascular network surrounding alveoli in a human subject suffering from an infection with a respiratory virus. It is moreover preferred that the viral infection is caused by a virus requiring a specific receptor and a more ubiquitous binding partner present on at least a percentage of lung alveolar cells. In one preferred embodiment, it is preferred that the specific receptor is ACE-2. In another preferred embodiment, it is preferred that the more ubiquitous binding partner is a glycoprotein comprising a sialic acid residue. It is, in particular, preferred that the ubiquitous binding partner binds with a fMLF-like amino acid sequence, for example, wherein the sequence at least comprises a membrane-proximal-external-region (MPER, herein also identified as fusogenic sequence(SEQ ID NO:1)). It is, in particular, preferred that the virus is a coronavirus, in particular, a coronavirus with an MPER as identified in FIG. 11, more in particular, at least comprising a fusogenic sequence as identified in FIG. 12. It is most preferred that the 1VIPER at least comprises amino acid sequence KWPWIWL (amino acids identified herein by one-letter code) (SEQ ID NO:1). In a further preferred embodiment, the disclosure provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, such as with a virus, wherein the coronavirus is the COVID-19 virus (SARS-COV-2) or a mutant thereof.

In a first embodiment, the disclosure provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, such as with a virus. In a preferred embodiment, the disclosure provides the method for reducing the gas diffusion distance between lung-alveoli and the vascular network surrounding alveoli in a human subject suffering from a respiratory infection, allowing reduction of fluid in alveoli and/or allowing improved oxygen supply to the subject's body. In a preferred embodiment, the substance comprises an AQGV-peptide(SEQ ID NO:2), an LQGV-peptide(SEQ ID NO:3), or a functional analogue of either. In a preferred embodiment, the disclosure provides the method for reducing the gas diffusion distance between lung-alveoli and the vascular network surrounding alveoli in a human subject suffering from an infection with a respiratory virus. It is moreover preferred that the viral infection is caused by a virus requiring a specific receptor and a more ubiquitous binding partner present on at least a percentage of lung alveolar cells. In one preferred embodiment, it is preferred that the specific receptor is ACE-2. In another preferred embodiment, it is preferred that the more ubiquitous binding partner is a glycoprotein comprising a sialic acid residue. It is, in particular, preferred that the ubiquitous binding partner binds with a fMLF-like amino acid sequence, for example, wherein the sequence at least comprises a membrane-proximal-external-region (MPER, herein also identified as fusogenic sequence). It is, in particular, preferred that the virus is a coronavirus, in particular, a coronavirus with an MPER as identified in FIG. 11, more in particular, at least comprising a fusogenic sequence as identified in FIG. 12. It is most preferred that the MPER at least comprises amino acid sequence KWPWIWL ((SEQ ID NO:1)amino acids identified herein by one-letter code). In a further preferred embodiment, the disclosure provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, such as with a virus, wherein the coronavirus is the COVID-19 virus (SARS-COV-2) or a mutant thereof.

The disclosure also provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, wherein the substance is administered intravenously to the subject, preferably at a rate of at least 75 mg/kg/hour, or more preferably at least 90 mg/kg/hour. It is moreover preferred that the substance is administered intermittently. It is moreover preferred that during treatment the subject is monitored for hemodynamic stability. The disclosure also provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, wherein the substance is administered intravenously to the subject, the method further comprising administering an antiviral agent, such as remdesivir (GS-5734), an inhibitor of the viral RNA-dependent, RNA polymerase, to the subject. The disclosure also provides a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, wherein the substance is administered intravenously to the subject, the method further comprising administering an anti-inflammatory agent, such as dexamethasone or an interleukin-6 signaling inhibitors such as tocilizumab, to the subject.

The disclosure also provides a pharmaceutical formulation for use in a method for reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, wherein the substance is administered intravenously to the subject, preferably at a rate of at least 75 mg/kg/hour, or more preferably at least 90 mg/kg/hour. It is moreover preferred that the substance is administered intermittently. It is moreover preferred that during treatment the subject is monitored for hemodynamic stability. The disclosure also provides a pharmaceutical formulation for use in reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, wherein the substance is administered intravenously to the subject, the method further comprising administering an antiviral agent, such as remdesivir (GS-5734), an inhibitor of the viral RNA-dependent, RNA polymerase, to the subject. The disclosure also provides a pharmaceutical formulation for use in reducing the permeability of an endothelial layer of a blood vessel in a subject, the method comprising providing to the endothelial layer a substance that reduces the ratio of Angiopoietin-2 to Angiopoietin-1 at the site of increased permeability as a result of an infection, wherein the substance is administered intravenously to the subject, the method further comprising administering an anti-inflammatory agent, such as dexamethasone or an interleukin-6 signaling inhibitors such as tocilizumab, to the subject.

The disclosure also provides a pharmaceutical formulation for use according to the invention, comprising an AQGV-peptide(SEQ ID NO:2), an LQGV-peptide(SEQ ID NO:3), or a functional analogue of either and an excipient suitable for parenteral administration.

When a human subject is suffering from an infection, in particular, a viral infection, more in particular, a viral respiratory infection, many effects are seen that influence hemodynamic stability. One of the effects seen is increased permeability of blood vessels leading to fluid leakage from blood vessels into the intercellular spaces, and vice versa, resulting in aggravated and traumatic damage to the lung, and other organs. Typical signs of such suffocating damage induced by leakage from vessels include increased extra-cellular fluid in lungs with fluid overflow into alveoli. Also, thrombosis may be seen, in particular, leading to (deep) venous thrombosis ((D)VT) and pulmonary embolism (PE). Particularly in respiratory infections, this all may lead to an increased diffusion distance for gasses such as oxygen and carbon dioxide to traverse the distance between alveoli and blood, and to hypoxemia. Oxygen and carbon dioxide both need to pass through a thin layer in the lungs called the alveolar-capillary membrane. This is the thin layer between the small air sacks in the lung (the alveoli) and the smallest blood vessels that travel through the lungs (lung-capillaries). How well oxygen is inhaled and can pass (diffuse) from the alveoli into the blood, and how well carbon dioxide can pass from the blood capillaries back into the alveoli to be exhaled, depends on how thick (swollen) this membrane is, and how much surface area is available for the transfer to take place.

This problem is aggravated in people already suffering from limited oxygen availability through underlying disease. Diffusing capacity may be low if there is less surface area available for the transfer of oxygen and carbon dioxide, for example, with emphysema, or if a lung or part of it is removed for lung cancer, or PE and or pre-existing cardiovascular and metabolic issues and obesity.

Also, diffusing capacity may be low if lung disease is present that causes the membrane to be thicker, for example, in chronic lung disease such as pulmonary fibrosis, as, for example, seen with COPD, and with sarcoidosis. The present disclosure is particularly useful for such patients having only partial lung capacity.

Acute disease can also result in low diffusing capacity, for example, in aggravated viral respiratory infections with injury to the lung, often the permeability of lung-capillaries is increased generating a flux of fluid from the capillaries into the thin layer of extra-cellular-matrix separating alveoli from capillaries, with intercellular fluid retention therewith thickening (swelling) the membrane through accumulation of fluid in the extra-cellular-matrix (interstitium) separating alveolar cells from vascular cells. In such lung injury patients and patients with aggravated infectious airway infections, plasma levels of biomarkers of endothelial activation, as may be measured by ELISA, are often predictive of mortality and morbidity. In particular, the concentration of angiopoietin-2 relative to angiopoietin-1 (Ang-2/Ang-1) may be a useful biologic marker of mortality in acute lung injury (ALI) patients. Ang-2/Ang-1 is found significantly higher in patients who died of lung injury [p=0.01; Crit. Care Med. 2010 September; 38(9): 1845-1851.]. In a multivariable analysis stratified by dead space fraction, Ang-2/Ang-1 was an independent predictor of death with an adjusted odds ratio of 4.3 (95% CI 1.3-13.5, p=0.01) in those with an elevated pulmonary dead space fraction (p=0.03 for interaction between pulmonary dead space fraction and Ang-2/Ang-1).

Similarly, D-dimer plasma levels may be used to follow a patient's health status in aggravated viral respiratory infections with injury to the lung and endothelial activation. D-dimer, the lysis product of cross-linked fibrin indicates fibrinolysis in response to clotting activation and fibrin formation (doi.org/10.1111/jth.12075). D-dimer levels are evident in febrile and convalescent phases typically following viral infections that affect vascular endothelial cells and associate with endothelial activation and plasma leakage. D-dimer assays can vary in sensitivity depending on the lab-specific type drawn, and not all labs report the same units providing various acceptable ranges for the results. There are many things that can cause elevated D-dimer beyond venous thromboembolism (VTE), such as age or pregnancy. The D-dimer half-life of 8 hours results in elevated levels for approximately 3 days after the inciting event. Quantitative D-dimer holds a sensitivity of 94% to 98%, yet only specificity of 50% to 60%. This allows to utilize it as a screening tool but requires clinical evidence from the history and physical examination, preferably with intermittent repeated testing to confirm the diagnosis or follow a patient's health status.

Thus, the disclosure in one aspect provides a method for reducing the permeability of an endothelial layer of a blood vessel comprising providing to the layer a substance reducing the ratio of angiopoietin-2 to angiopoietin-1 at the site of increased permeability as a result of an infection. In one embodiment this method serves to reduce the gas diffusion distance (or at least to prevent increasing the diffusion distance) between lung-alveoli and the vascular network surrounding alveoli in a human subject suffering from a respiratory infection, in particular, patients with underlying disease causing limited oxygen availability. Reduced vascular permeability in patients suffering vascular leakage is generally associated with reduced D-dimer levels. According to the invention, in one embodiment the substances to be used in the methods according to the disclosure include peptides that influence hemodynamics, particularly by influencing gap junctions between the cells. Such peptides include AQGV (SEQ ID NO:2) and functional analogues thereof. A functional analogue is defined as a substance that provides the same or a similar function (in kind, not necessarily in amount). Basically, any substance that decreases permeability of the vascular system may be used according to the present invention. For one, tetrapeptide AQGV ((SEQ ID NO:2) herein also referred to as EA-230) has surprisingly been found to modulate vascular permeability to the good. In particular, EA-230 significantly improves hemodynamic stability in humans, even in the absence of inflammatory activity of the patient. Permeability governs the amount of fluid leaking from blood vessels. Administration of fluid therapy generally increases leakage. Based on Phase II trial patient observations, a significant reduction of adverse fluid retention (fluid leakage) was found in patients treated with EA-230 (p=0.03). Throughout clinical trials, EA-230 was shown to be safe and well tolerated. EA-230 shows significant improvements in patient recovery, over placebo patient. EA-230 treated patients are released faster from intensive care (p=0.0232) and hospital (p=0.0015). EA-230 improves hemodynamic stability (p=0.006) and kidney function (p=0.003). Long-term patient recovery was significantly improved by EA-230. By improving vascular permeability, EA-230 can be used to reduce the infection-associated occurrence of adverse fluid in the lungs, reduce hypoxemia, reduce PE, and therewith also reduce ventilator use with its detrimental systemic effects, in particular, in viral respiratory infections such as caused by influenza viruses and in particularly by coronaviruses. Thus, provided is, in particular, a method according to the disclosure wherein the active substance to control hemodynamic stability comprises an AQGV peptide(SEQ ID NO:2). A functional and/or structural AQGV analogue according to the disclosure may be selected from the group consisting of peptides comprising a tetrapeptide selected from the group of AQLP (SEQ ID NO:4), PLQA (SEQ ID NO:5), LQGV (SEQ ID NO:3), LAGV (SEQ ID NO:7), PQVG (SEQ ID NO:8), PQVA (SEQ ID NO:9), PQVR (SEQ ID NO:10), VGQL (SEQ ID NO:11), LQPL (SEQ ID NO:12), RQGV (SEQ ID NO:13), LQVG (SEQ ID NO:14), LQGA (SEQ ID NO:15), LQGR (SEQ ID NO:16), AQGA (SEQ ID NO:17), QPLA (SEQ ID NO:18), PQVP (SEQ ID NO:19), VGQA (SEQ ID NO:20), QVGQ (SEQ ID NO:21), VGQG (SEQ ID NO:22) or other permutations of peptides of 4-12 amino acids constituted of particularly the amino acids of the above tetrapeptides. The disclosure further provides a method wherein the viral infection is caused by a virus requiring a specific receptor and a more ubiquitous binding partner present on at least a percentage of lung alveolar cells. In one preferred embodiment, the viral infection is caused by a coronavirus wherein the specific receptor is ACE-2, in particular, wherein the coronavirus is SARS-Cov-2 or a mutant or analogue thereof. Other coronavirus infections that may be treated according to the disclosure carry specific receptor DPP4 (such as with MERS corona virus) or APN (aminopeptidase N). Also preferred is a method wherein the more ubiquitous binding partner is a glycoprotein comprising a sialic acid residue. It is, in particular, preferred that the ubiquitous binding partner binds with a fMLF-like amino acid sequence, for example, wherein the sequence at least comprises a membrane-proximal-external-region (MPER, herein also identified as fusogenic sequence). Such a method according to the disclosure is, in particular, provided wherein the virus is a coronavirus. It is, in particular, preferred that the virus is a coronavirus, in particular, a coronavirus with an MPER as identified in FIG. 11, more in particular, at least comprising a fusogenic sequence as identified in FIG. 12. Alternatively, the more ubiquitous binding partner is a glycoprotein comprising a sialic acid residue as recognized by influenza virus. The combination of a specific and a more ubiquitous binding/infection site on a cell is typical for coronaviruses, with their typical effect on the hemodynamics as disclosed herein. In a further preferred embodiment, provided is a method wherein the AQGV peptide or related substance is administered intravenously, preferably at a rate of at least 75 mg/kg/hour, more preferably at least 90 mg/kg/hour. It is, in particular, useful to administer the AQGV peptide or related substance intermittently. Preferred use is dosing for 2-4 hours at at least 90 mg/kg/hour, then reducing to 30 mg/kg/hour for 2-4 hours, or for as long as it takes to monitor the patients response to treatment by clinical or laboratory diagnosis, or stop administering the substance for 1-2 hours until diagnostic studies such as point-of-care testing have completed, and then resume treatment with 2-4 hours at at least 90 mg/kg/hour. It is preferred that the monitoring comprises studying the subject for hemodynamic stability and/or fibrinolysis. Treatment with AQGV peptide (SEQ ID NO:2) according to the disclosure may further comprise administering an antiviral agent. The disclosure also provides a pharmaceutical formulation comprising an AQGV peptide (SEQ ID NO:2) or related substance (preferably a functional analogue) for use in a method according to the invention, or a pharmaceutical formulation for use according to the invention, comprising an AQGV peptide (SEQ ID NO:2), or a functional analogue thereof and an excipient suitable for parenteral administration.

Often, the human subject or patient experiencing reduced diffusion, may be admitted into an intensive care unit (ICU) where vital signs are monitored. The patient receives medical treatment to allow the patient to recover and when vital signs are within acceptable boundaries, the patient can be released from ICU and admitted into standard hospital care. When the patient has shown to be stable at standard care, the patient can be released from the hospital and returns home. Subsequently, a patient can be readmitted into the hospital should the need arise because, e.g., the condition or infection status of the patient worsens. Any improvement on the health and recovery of a patient affecting the length of stay of a patient in the ICU, the length of stay in standard care at the hospital and/or patient re-admittance, provides for a significant benefit to patients. Hence, any means and methods according to the disclosure that improve the health and, in particular, speed of recovery of a patient through use of the peptide compounds disclosed herein are of interest.

Upon testing in a clinical trial aimed at assessing the safety and tolerability of an AQGV (SEQ ID NO:2) peptide (also referred to as EA-230 herein) and its immunomodulatory effects, the peptide was found to be safe but, unexpectedly, no immunomodulatory effects were observed under the test circumstances when comparing treated patients as compared with control subjects. Instead of observing immunomodulatory effects, the current inventors surprisingly found that upon analysis of the data obtained in the clinical trial, new and highly advantageous properties could be attributed to the AQGV (SEQ ID NO:2) peptide, which have not been observed before. These properties are apparently independent from known and observed immunomodulatory effects.

Hence, the current disclosure relates to the use of an AQGV (SEQ ID NO:2) peptide, and analogues thereof, for improving the clinical parameters of human patients admitted into hospital and/or intensive care such that the time period between admittance and release from hospital and/or intensive care can be shortened. In one embodiment, the use of an AQGV (SEQ ID NO:2) peptide, and analogues thereof, is for use in a medical treatment for modifying hemodynamics in human subjects. In a further embodiment, the use in human subjects for modifying hemodynamics, involves a reduction of reducing undesired fluid retention and/or a reduced use of vasopressive agents in the human subject. In another embodiment, the use of an AQGV (SEQ ID NO:2) peptide, and analogues thereof, is for use in human subjects having impaired lung function.

In one embodiment, an AQGV (SEQ ID NO:2) peptide, or a functional analogue thereof, is provided for use in a method of treatment of a human subject, wherein the use comprises a treatment for modifying hemodynamics in the human subject. Hemodynamics involves the dynamics of blood flow, i.e., the physical factors that govern blood flow through the human body. Hemodynamics in human patients can be monitored by measuring, e.g., blood pressure and/or the fluid balance. When blood pressure is low and/or the fluid balance disturbed in a human patient, vasopressors, or inotropes may be used and/or fluid administered, e.g., intravenously. Inotropes and vasopressors are biologically and clinically important vasoactive medications that originate from different pharmacological groups and act at some of the most fundamental receptor and signal transduction systems in the body. More than 20 such agents are in common clinical use, yet few reviews of their pharmacology exist outside of physiology and pharmacology textbooks. Despite widespread use in critically ill patients, understanding of the clinical effects of these drugs in pathological states is poor. Adverse effects of vasopressors and inotropes depend on the mechanism of action. For the medications that have beta stimulation, arrhythmias are one of the most common adverse effects that one would like to reduce.

The current inventors have found that by using an AQGV (SEQ ID NO:2) peptide, or a functional analogue thereof, the hemodynamics in human patients post-trauma (e.g., viral infection) was significantly improved as shown by, e.g., a reduced use of vasopressors and/or an improved fluid balance in human patients. The use of an AQGV (SEQ ID NO:2) peptide, or a functional analogue thereof, as described herein thus improves the hemodynamic stability in human patients. Modifying or optimizing hemodynamics in human subjects is of importance post-injury, when, e.g., human subjects have suffered infection, trauma and/or blood loss. Hence, the AQGV (SEQ ID NO:2) peptide, or an analogue thereof, can advantageously be used in hemodynamic therapy. Hemodynamic therapy comprises the optimization of hemodynamics in patients in goal-directed hemodynamic therapy. Such therapies can include therapeutic interventions such as fluid management in patients and/or the use of vasopressors.

AQGV (SEQ ID NO:2) functional analogues are defined herein as peptides exerting analogous effect or function as the AQGV (SEQ ID NO:2) peptide as described herein, in kind not necessarily in amount. The AQGV (SEQ ID NO:2) peptide has a length of 4 amino acids. An AQGV (SEQ ID NO:2) functional analogue may have sequence identity, i.e., comprising at least part or the whole of the AQGV (SEQ ID NO:2) peptide. Preferably, such an AQGV (SEQ ID NO:2) functional analogue is a structural analogue of the AQGV (SEQ ID NO:2) peptide. A preferred structural analogue may be an LQGV (SEQ ID NO:3) peptide. Structural analogues of the AQGV (SEQ ID NO:2) peptide may be selected from peptides comprising amino acids selected from the group of amino acids alanine (in one letter code: A), glutamine (Q), glycine (G), valine (V), leucine (L), proline (P) and arginine (R). In a preferred embodiment, provided is for a AQGV (SEQ ID NO:2) structural analogue, that comprises at least 50%, more preferably at least 75%, most preferably at least 100% amino acids selected from the group of autophagy-inhibiting amino acids alanine (in one letter code: A), glutamine (Q), glycine (G), valine (V), leucine (L), proline (P), and arginine (R). Preferably, a structural analogue of the AQGV (SEQ ID NO:2) peptide has a length in the range of 4-12 amino acids. Preferably, such a structural analogue is a linear peptide. Suitable structural analogues of AQGV (SEQ ID NO:2) may have a length less than 4, e.g., of 3, however such lengths may require higher doses of such peptides because the half-life of such peptides will be shorter and thus less preferred. Longer structural analogues, e.g., longer than 12 residues, are less preferred because of potential immunogenicity of such longer peptides. A structural AQGV (SEQ ID NO:2) analogue according to the disclosure may be selected from the group consisting of peptides comprising a tetrapeptide selected from the group of AQLP (SEQ ID NO:4), PLQA (SEQ ID NO:5), LQGV (SEQ ID NO:6), LAGV (SEQ ID NO:7), PQVG (SEQ ID NO:8), PQVA (SEQ ID NO:9), PQVR (SEQ ID NO:10), VGQL (SEQ ID NO:11), LQPL (SEQ ID NO:12), RQGV (SEQ ID NO:13), LQVG (SEQ ID NO:14), LQGA (SEQ ID NO:15), LQGR (SEQ ID NO:16), AQGA (SEQ ID NO:17), QPLA (SEQ ID NO:18), PQVP (SEQ ID NO:19), VGQA (SEQ ID NO:20), QVGQ (SEQ ID NO:21), and VGQG (SEQ ID NO:22).

Vasopressors are a class of drugs that can elevate low blood pressure. Some vasopressors act as vasoconstrictors, other vasopressor sensitize adrenoreceptors to catecholamines-glucocorticoids, and another class of vasopressors can increase cardiac output. Whichever vasopressor is used, the current disclosure allows for a reduction in the use of vasopressors. A reduction in the use of vasopressors involves a reduction of the duration of vasopressor use and/or a reduction of the dosage of the vasopressor. Examples of vasopressors are, e.g., epinephrine, noradrenaline, phenylephrine, dobutamine, dopamine, and vasopressin. Fluid management in patients involves monitoring, e.g., oral, enteral, and/or intravenous intake of fluids and fluid output (e.g., urine) and subsequently managing fluid intake, e.g., in case of an observed fluid retention (i.e., the fluid intake exceeds fluid output). Strikingly, the use of the AQGV (SEQ ID NO:2) peptide, or an analogue thereof, can reduce fluid retention. Hence, the AQGV (SEQ ID NO:2) peptide, or analogue thereof, can be used in addition to known interventions that are to improve the hemodynamics in human patients, thereby resulting in a faster improvement in hemodynamics as compared with not using an AQGV (SEQ ID NO:2) peptide, or an analogue thereof.

In another embodiment, an AQGV (SEQ ID NO:2) peptide, or a functional analogue thereof, is provided for use in the treatment of a human subject having impaired lung function. In a further embodiment, the impaired lung function is acute lung injury. In one embodiment, an AQGV peptide, or a functional analogue thereof, is provided for use in the treatment of a human subject for improving lung function. Lung function can also be assessed by measuring hypoxemia, or by determining the alveolar-arterial gradient (A-aO2, or A-a gradient). Assessing A-a gradient to assess lung function in humans is standard clinical practice (e.g., by determining the difference between the alveolar concentration (A) of oxygen and the arterial (a) concentration of oxygen. It is used in diagnosing the source and degree of hypoxemia. The A-a gradient helps to assess the integrity of the alveolar capillary unit. Improvements in lung function as compared with not receiving the AQGV (SEQ ID NO:2) peptide can include progressing to a lung function stage to a less severe stage (e.g., a patient progressing from having lung injury to being at risk of lung injury or having no lung injury). Irrespective of what assessment is made, the use of the AQGV (SEQ ID NO:2) peptide, or analogue thereof, can improve lung function in humans having lung injury and/or an impairment of lung function in subjects absent of immunomodulatory effects.

The use of the AQGV (SEQ ID NO:2) peptide allows for improving lung function, however, it can also prevent a reduction and/or an impairment of lung function. Accordingly, lung injury with hypoxemia may be prevented. Hence, in one embodiment, the use of the AQGV (SEQ ID NO:2) peptide, or analogue thereof, allows to maintain lung function in human patients. Hence, the use of the AGQV peptide, or analogue thereof allows to protect lung function in human patients. In another embodiment, the use of the AQGV (SEQ ID NO:2) peptide, or analogue thereof, allows to prevent a reduction and/or impairment of lung function in human patients. For example, a human patient that can be classified as having no lung injury, or being at risk of having lung injury (such as with COVID-19), may receive treatment with the AQGV (SEQ ID NO:2) peptide, whereby such a patient may maintain its status instead of progressing to a (more severely) reduced lung function. Hence, human patients that are at risk of developing lung injury, e.g., due to (induced) trauma, such as infection, may, as a result of receiving treatment with the AQGV (SEQ ID NO:2) peptide, or analogue thereof, maintain their lung function status.

In another embodiment, an AQGV (SEQ ID NO:2) peptide, or a functional analogue thereof, is provided for use in the treatment of a human subject having impaired lung function whereby the use comprises modifying hemodynamics in the human subject. As treatment of lung function and treatment of hemodynamic stability can now be linked, the use of an AQGV (SEQ ID NO:2) peptide, or a functional analogue thereof, in accordance with the disclosure can advantageously be used to protect lung function and/or improve lung function, and modifying hemodynamics. Such combined use resulting, e.g., in improved and/or maintained lung function and a reduction in the use of vasopressors and/or improved fluid management in human subjects.

In a further embodiment, the current disclosure provides for a reduced use of vasopressive agents. The use of vasopressive agents can be reduced by reducing the duration of the use of vasopressive agents. The use of vasopressive agents can be reduced by reducing the amount of vasopressive agents (e.g., reducing amount per dosage and/or increasing time interval between administrations). The use of vasopressive agents can be reduced by reducing the amount of vasopressive agents and the duration of the use of vasopressive agents. By reducing the use of vasopressive agents, human subjects advantageously recover more quickly as compared with human subjects not receiving AQGV (SEQ ID NO:2), or an analogue thereof.

In another embodiment, the use of an AQGV (SEQ ID NO:2) peptide, or a functional analogue thereof, reduces adverse fluid retention in the human subject. Fluid retention can occur in human subjects, symptoms of which, e.g., include weight gain and edema. Fluid retention can be the result of reduced lung function and/or impaired hemodynamics. Hence, because the use of AQGV can affect lung function and/or hemodynamic stability in human subjects, the use of AQGV (SEQ ID NO:2) can affect fluid retention as well. Fluid retention can be the result of leaky capillaries. Hence, the use of AQGV (SEQ ID NO:2), and analogues thereof, may have an effect on the leakiness (permeability) of capillaries, reducing leakage of plasma from the blood to peripheral tissue and/or organs. Most preferably edema can be reduced and/or avoided by the use of AQGV (SEQ ID NO:2). Such may also be referred to as adverse fluid retention as it has an adverse effect on the patient. Whichever is the cause of fluid retention, the use of an AQGV (SEQ ID NO:2) peptide, or a functional analogue thereof can improve fluid retention dynamics in human subjects thereby alleviating symptoms associated with fluid retention such as weight gain and edema, which subsequently can reduce the use of diuretics.

In another embodiment, the use of the AQGV (SEQ ID NO:2) peptide, or a functional analogue thereof, in accordance with the invention, is not restricted to patients having lung injury and/or requiring hemodynamic therapy. The use of an AQGV (SEQ ID NO:2) peptide, or a functional analogue thereof, in accordance with the invention, includes the treatment of human patients that are believed to be at risk of having lung injury and/or anticipated to require hemodynamic therapy. Such human patients include patients that are to be admitted, or are expected to be admitted, into intensive care. Hence, the use of the AQGV (SEQ ID NO:2) peptide, or a functional analogue thereof, includes a use for trauma, such as infection, as exemplified, e.g., in the examples. The use of the AQGV (SEQ ID NO:2) peptide for trauma, such as infection, may be before, but is typically during and/or after infection. It may be preferred that the use of the AQGV (SEQ ID NO:2) peptide, or an analogue thereof, is during infection with a virus. The use of AQGV (SEQ ID NO:2) peptide as is provided herein is, in particular, useful in patients subjected to a long duration of mechanical ventilation, i.e., longer than 2.5 hours. Hence, in a further embodiment, the use of the AQGV (SEQ ID NO:2) peptide, or an analogue thereof, is during a mechanical ventilation of longer than 2.5 hours and wherein the AQGV (SEQ ID NO:2) peptide or analogue thereof is administered during the mechanical ventilation. In another, or further, embodiment, the use of an AQGV (SEQ ID NO:2) peptide, or a functional analogue thereof, for use in accordance with the disclosure is for use is in a human subject having COVID-19. It is well known that shortening the duration of mechanical ventilation is highly correlated with recovery and prevention of re-admittance of patients.

Preferably, the use of the AQGV (SEQ ID NO:2) peptide, or a functional analogue thereof, in accordance with the disclosure and as described above, involves the administration of the peptide into the bloodstream. It is understood that administration into the bloodstream comprises, e.g., intravenous administration or intra-arterial administration. A constant supply of AQGV (SEQ ID NO:2) peptide, or an analogue thereof, is preferred, e.g., via an infusion wherein the AQGV (SEQ ID NO:2) peptide, or analogue thereof, is comprised in a physiological acceptable solution. Suitable physiological acceptable solutions may comprise physiological salt solutions (e.g., 0.9% NaCl) or any other suitable solution for injection and/or infusion. Such physiological solutions may comprise further compounds (e.g., glucose etc.) that may further benefit the human subject, and may also include other pharmaceutical compounds (e.g., vasopressors).

Preferably, the AQGV (SEQ ID NO:2) peptide is administered at a rate that is at least 50 mg/kg patient weight per hour (mg/kg/hr). Preferably the administration rate is at least 60 mg, at least 70, at least 80 or, most preferably, at least 90 mg/kg/hr. Preferably, the AQGV (SEQ ID NO:2) peptide is administered for at least 1 hour, more preferably at least 1.5 hours, most preferably at least 2 hours. Preferably, the administration of the AQGV (SEQ ID NO:2) peptide is at a rate of at least 70 mg/kg/hr. and administered for at least 1 hour, more preferably at least 1.5 hours, most preferably at least 2 hours. Preferably, the administration is during infection. More preferably, the administration is during essentially the entire duration of disease resulting from infection. Typically treatment will start after determination of a level of severity justifying the treatment. Thus the treatment may typically last from its detection until the absence of detectable infection or sufficient recovery to allow for end of treatment.

As shown in the example section, the mean arterial maximum concentrations (mean Cmax) as determined in vivo in humans for EA-230 in the Phase II clinical trial was 30500 ng/mL, in the range of 12500 to 57500 ng/mL. The mean venous Cmax found was 68400 ng/mL, in the range of 19600 to 113000 ng/mL. Hence, whichever means and methods are used for administration of EA-230 (or AQGV), preferably, means and methods that allow obtaining an arterial Cmax in the range of 10,000 to 60,000 ng/mL and/or a venous Cmax in the range of 15000 to 120000 ng/ml can be contemplated. Thus, the route of administration may not be necessarily be restricted to intravenous administration, but may include other routes of administration resulting in similar venous and/or arterial Cmax concentrations.

In another embodiment, an AQGV (SEQ ID NO:2) peptide, or a functional analogue thereof, is provided for any use in accordance with the disclosure as described above, wherein the human subject is admitted to intensive care, and wherein the use improves parameters measured of the human subject, the parameters of the human subject typically those being determined to assess whether the patient needs to remain in intensive care or not. As shown above, parameters that are assessed when a human patient is in intensive care include parameters related to lung function and hemodynamics. In any case, the use of the AQGV (SEQ ID NO:2) peptide, or analogue thereof, is to improve such parameters to thereby reduce the length of stay in the intensive care unit. Not only does the use of the AQGV (SEQ ID NO:2) peptide, or analogue thereof reduce the length of stay in the intensive care, the effect of the use of the AQGV (SEQ ID NO:2) peptide, or analogue thereof, also reduces the length of stay in the hospital and reduces re-admittance into the hospital.

In any case, the use of the AQGV (SEQ ID NO:2) peptide, or a functional analogue thereof has a profound effect on lung function and/or hemodynamics in human subjects thereby advantageously benefiting human subjects when, e.g., suffering from induced trauma, e.g., when undergoing mechanical ventilation. Hence, in one embodiment, the use of the AQGV (SEQ ID NO:2) peptide, or a functional analogue thereof, is for use in patients subjected to mechanical ventilation. In another embodiment, the use of the AQGV (SEQ ID NO:2) peptide, or a functional analogue thereof, is for use in human patients experiencing or thought to be experiencing COVID-19 or a similar infection.

The disclosure relates to a distinct and new class of drugs: autophagy-inhibiting compounds that comprise peptides and/or amino acids that target the nutrient sensing system of the mechanistic target of rapamycine, mTOR and inhibit autophagy. Upon testing formyl-peptide related signaling effects of an autophagy-inhibiting AQGV (SEQ ID NO:2) peptide the peptide was found to unexpectedly attenuate p38/p38-MK2-HSP27 and/or PI3K/AKT/mTOR pathways that govern signal cytoskeleton contraction in modulating vascular permeability. Hence, the current disclosure relates to the use of an autophagy-inhibiting peptide herein also referred to as an AQGV (SEQ ID NO:2) peptide, and analogues (functional equivalents) thereof, for improving vascular permeability.

Without being bound by theory, the effect of the AQGV (SEQ ID NO:2) peptide, or a functional analogue thereof, may have an effect on vasoconstriction. Vasoconstriction involves the narrowing of the blood vessels resulting from contraction of the muscular wall of the vessel. Hence, in one embodiment, the use of an AQGV (SEQ ID NO:2) peptide, or a functional analogue thereof, in accordance with the invention, involves inducing vasoconstriction.

The disclosure also provides a method for identifying a peptide capable of reducing p38 MAPK kinase activity, comprising providing cells with a peptide comprising amino acids, the amino acids for at least 50%, preferably at least 60%, more preferably at least 70%, more preferably at least 80%, more preferably at least 90%, more preferably 100%, amino acids selected from the group of alanine (in one letter code: A), glutamine (Q), glycine (G), valine (V), leucine (L), isoleucine (I), proline (P) and arginine (R), providing the cells with fMLP and detecting phosphorylation of p38 MAPK in the absence and presence of the peptide at an appropriate time interval, preferably in the order of minutes, most preferably from about half a minute to about 5 minutes, e.g., 30 to 600 seconds after provision of fMLP, and comparing the results to determine the peptide's effect on the phosphorylation. Having tested the autophagy-inhibiting AQGV (SEQ ID NO:2) peptide, FPR-activation of FPR-expressing cells with prototype FPR-ligand fMLP was detected to cause rapidly induced and significant (p<0.05; p38 from 60 to 600 sec, PKB at 600 sec) changes in phosphorylation status of PKB (also known as AKT) (FIG. 10a) and p38 MAPK kinases (FIG. 10c), but not (or not detected) in STAT3, JNK (FIG. 10b) and P42/p44MAPK/ERK1,2 (FIG. 10d) kinases.

Therewith the disclosure also provides a method for identifying a peptide capable of reducing PI3K/AKT/mTOR activity, comprising providing cells with a peptide consisting of amino acids, the amino acids for at least 50%, preferably at least 60%, more preferably at least 70%, more preferably at least 80%, more preferably at least 90%, more preferably 100%, amino acids selected from the group of alanine (in one letter code: A), glutamine (Q), glycine (G), valine (V), leucine (L), isoleucine (I), proline (P) and arginine (R), providing the cells with fMLP and detecting phosphorylation of PKB (AKT) in the absence and presence of the peptide at an appropriate time interval, preferably in the order of minutes, most preferably from about half a minute to about 5 minutes, e.g., 30 to 600. AQGV (SEQ ID NO:2) peptide effects on p38 MAPK (FIG. 10c) are already detected at 30 seconds after FPR-stimulation, AQGV (SEQ ID NO:2) peptide effects on PKB(AKT) follow (FIG. 10a) in a bi-phasic pattern at 300 sec. Both AQGV peptide effects on p38 and PKB-mediated signaling last for the full 600 seconds tested whereas the other kinases tested were not affected throughout. As this acute and specific response to treatment shows specific and rapid effects of autophagy-inhibiting-AQGV peptide on p38 signaling in the context of regulation of the PI3K/AKT/mTOR pathway, the pathway is governing the balance between proteolysis and proteogenesis regulating cytoskeleton changes affecting vascular permeability. Such activities are not detected in STAT3, JNK (FIG. 10b) and P42/p44MAPK/ERK1,2 (FIG. 10d) kinases tested with AQGV (SEQ ID NO:2) peptide. It is shown that AQGV (SEQ ID NO:2) peptide reduces p38 MAPK kinase activated changes as well as reduces PI3K/AKT/mTOR activated induced changes in cell cytoskeleton reorganization affecting endothelial cell contraction and adverse vascular permeability.

The disclosure also provides a method for identifying a peptide capable of reducing PI3K/AKT/mTOR activity, comprising providing cells with a peptide consisting of amino acids, the amino acids for at least 50%, preferably at least 60%, more preferably at least 70%, more preferably at least 80%, more preferably at least 90%, more preferably 100%, amino acids selected from the group of alanine (in one letter code: A), glutamine (Q), glycine (G), valine (V), leucine (L), isoleucine (I), proline (P) and arginine (R), providing the cells with fMLP and detecting phosphorylation of PKB (AKT) in the absence and presence of the peptide at an appropriate time interval, preferably in the order of minutes, most preferably from about half a minute to about 5 minutes, e.g., 30 to 600 seconds after provision of fMLP, and comparing the results to determine the peptide's effect on the phosphorylation. Identified AQGV (SEQ ID NO:2) peptide is useful and capable of addressing adverse vascular permeability, such as manifested by edema with vascular leakage, adverse leukocyte extravasation and hypotension in human subjects.

The disclosure also provides a method for identifying a peptide capable of reducing PI3K/AKT/mTOR activity, comprising providing cells with a peptide consisting of amino acids, the amino acids for at least 50% selected from the group of alanine (in one letter code: A), glutamine (Q), glycine (G), valine (V), leucine (L), isoleucine (I), proline (P) and arginine (R), providing the cells with fMLP and detecting phosphorylation of PKB (AKT) in the absence and presence of the peptide at an appropriate time interval, preferably in the order of minutes, most preferably from about half a minute to about 5 minutes, e.g., 30 to 600 seconds after provision of fMLP, and comparing the results to determine the peptide's effect on the phosphorylation. The disclosure therewith also provides method for identifying a peptide capable of reducing cytoskeleton reorganization, comprising providing cells with a peptide consisting of amino acids, the amino acids for at least 50% selected from the group of alanine (in one letter code: A), glutamine (Q), glycine (G), valine (V), leucine (L), isoleucine (I), proline (P) and arginine (R), and providing the cells with fMLP and detecting phosphorylation of p38 MAPK and/or PKB (AKT) in the absence and presence of the peptide at an appropriate time interval, preferably in the order of minutes, most preferably from about half a minute to about 5 minutes after provision of fMLP, and comparing the results to determine the peptide's effect on the phosphorylation.

Typically, as the disclosure herein provides a molecular mode-of-action (MoA) of the group of autophagy-inhibiting peptides its effects do not necessarily depend on their exact sequence. Instead, their constituent amino acids provide common household, “no-danger or tissue-repair” signals to the nutrient-sensing system of mTOR; leading to inhibition of autophagy and resulting in resolve of disease. These tissue-repair signal molecules change the balance of proteogenesis versus proteolysis in a cell of and lead to resolve of disease in three steps:

Administered peptide or amino acid fragments thereof are for taken up by amino acid transport, PEPT1/2 transport, by common endocytosis, in the case of vascular cells by elastin receptor-mediated endocytosis or phagocytosis.

Internalized peptide is hydrolyzed and its amino acids are presented to the nutrient-sensing system of mTOR.

Particular amino acids inhibit autophagy, therewith inhibiting proteolysis and leading to proteogenic resolve and pharmaceutical effect.

Various peptides, either derived from breakdown of peptide hormones or assembled as novel synthetic peptide essentially comprising amino acids selected from the group of autophagy-inhibiting amino acids, meeting one or more of the characteristics of the above description have been shown in various animal models in mice or rats to provide potent resolve of excess or adverse—local or systemic-vascular permeability through effects on endothelial cells lining our vasculature. Several have or are being rationally developed further in various stages of human clinical trials. Exploiting the autophagy-inhibiting mechanism involved through future clinical application of these autophagy-inhibiting compounds and related peptide drugs provides an exciting novel avenue for the rational treatment of disease. However, with several autophagy-inhibiting peptide formulations for intravenous application, peptide solubility difficulties have been experienced that decrease availability of autophagy-inhibiting amino acids, necessitating providing stock solutions of peptide in cumbersome large volumes to avoid aggregation of peptide and loss of pharmaceutical effect.

It is a purpose of this disclosure to provide the autophagy-inhibiting amino acids in a most expedient way to a subject deemed in need thereof. Therewith, the disclosure provides a tartrate or a citrate of a, preferably recombinant or synthetic, autophagy-inhibiting peptide, the peptide having an amino acid sequence comprising at least 50%, more preferably at least 75%, most preferably at 100% amino acids selected from the group of autophagy-inhibiting amino acids alanine (A), glutamine (Q), glycine (G), valine (V), leucine (L), isoleucine (I) and proline (P). More preferably, the disclosure provides a stock solution, preferably aqueous, comprising a peptide-tartrate or a peptide citrate of a, preferably recombinant or synthetic, autophagy-inhibiting peptide, the peptide having an amino acid sequence comprising at least 50%, more preferably at least 75%, most preferably at 100% amino acids selected from the group of autophagy-inhibiting amino acids alanine (A), glutamine (Q), glycine (G), valine (V), leucine (L), isoleucine (I) and proline (P).

The disclosure provides a suitable solution for several autophagy-inhibiting peptides to mitigate aggregation of the peptides and identifies tartrate (from tartaric acid, preferably from (+)-tartaric acid) and more preferably citrate (from citric acid) as a suitable counter-ion, pharmaceutical excipient or anion of choice for preparing a salt of an autophagy-inhibiting peptide that is a neutral peptide as defined herein above. A variety of salts were screened herein to determine their influence on aggregation of neutral peptide according to the invention, indeed revealing that neutral peptide “salts out” of solution in an anion-specific and concentration-dependent manner. Aggregation points of such salts (the point of concentration below which aggregated peptide-salt tends to resolve) Peptide-sulfate, peptide-maleate, peptide-adenosine monophosphate and peptide-adenosine in aqueous solution were found to show aggravated aggregation in relation to peptide-acetate aggregation, whereas surprisingly tartrate, and more surprisingly peptide-citrate, showed (strongly) reduced aggregation in aqueous solution in comparison to peptide-acetate.

It is preferred that the autophagy-inhibiting peptide-salt according to the disclosure comprises <25% charged residues selected from the group K, H and R. It is more preferred that the autophagy-inhibiting peptide comprises <25% charged residues selected from the group D, K, R, H, and E. It is most preferred that the autophagy-inhibiting peptide-salt does not comprise residues selected from the group D, K, R, H, and E. It is furthermore preferred that the solution is an aqueous solution. In a most preferred embodiment, the solution is a so-called stock solution, preferably an aqueous stock solution. A stock solution generally is a concentrated solution of an active substance, herein autophagy-inhibiting peptide-salt, that will be diluted to some lower concentration for actual use of the substance, a so-called working solution.

Such lower concentration working solutions are, for example, infusion fluids, e.g., for intravenous or intra-abdominal use to which the peptide is added from the stock solution for administrating therapy to a patient, as often seen in critically ill patients, for example, at the intensive care of an hospital or at the battlefield. Under such conditions it is useful, and often considered a requisite, to have the active (peptide) drug available in a small (stock) volume for dilution into the infusion fluid. So-called stock solutions are generally provided and used to save solubilization and preparation time, conserve materials, reduce storage space, and improve the accuracy with which lower concentrated solutions are prepared to work with. Stock solutions of drugs are often prepared and then provided or stored for imminent intravenous use, for example, in critically ill patients. However, due to its by default higher peptide concentration, a stock solution with an autophagy-inhibiting peptide invariably runs higher risks on peptide drug aggregation than a final working solution. Stock solutions are generally prepared at a concentration well below an aggregation concentration of the salt in question (e.g., 40-50%) to prevent salt-out events under possibly prolonged storage at various ambient conditions. Risk of peptide aggregation (salting-out) is a phenomenon that the disclosure provides to avoid or mitigate herein with a stock solution according to the invention. Such stock solutions generally are diluted 10- to 100-fold, or more, to provide a suitable working solution. It is however also an object of the present disclosure to provide working solutions of the peptide-salts according to the invention. Particularly because in the application of the peptides of the disclosure relatively high amounts/concentrations of the peptide salts must be given, it is a prerequisite that the working solutions are far away from salting out points and yet are presented in a relatively small volume.

A variety of salts were screened herein to determine their influence on aggregation of neutral peptide, indeed revealing that neutral peptide “salts out” of solution in an anion-specific and concentration-dependent manner. Peptide-sulfate and peptide-maleate were found to show aggravated aggregation in relation to peptide-acetate aggregation, whereas surprisingly tartrate, and even more surprisingly peptide-citrate, showed (strongly) reduced aggregation in comparison to peptide-acetate.

The disclosure therewith contributes to improved solubility of this distinct and new class of drugs that is emerging: small autophagy-inhibiting peptides comprising amino acids that preferentially inhibit autophagy and target the nutrient sensing system of the mechanistic target of rapamycin, mTOR. Typically, peptides are defined as having 50 or less amino acids, for the purpose of this disclosure, proteins are defined as having >50 amino acids. An “autophagy-inhibiting peptide” herein is defined as a linear, branched or circular string of no longer than 50 amino acids that comprises a peptide sequence with at least 50%, more preferably at least 75%, most preferably 100% amino acids selected from the group of autophagy-inhibiting amino acids alanine (in one letter code: A), glutamine (Q), glycine (G), valine (V), leucine (L), proline (P), isoleucine (I) and arginine (R).

Molecular mode-of-action (MoA) of this group of peptides does not depend on their exact sequence. Instead, their constituent amino acids provide common household, “no-danger or tissue-repair” signals to the nutrient-sensing system of mTOR; leading to inhibition of autophagy and resulting in resolve of disease

In another embodiment, the disclosure provides a peptide, preferably a salt of an organic acid, such as a maleate, more preferably an acetate, more preferably a tartrate, most preferably a citrate of a, preferably recombinant or synthetic, autophagy-inhibiting peptide, the peptide having an amino acid sequence comprising 50%, more preferably at least 75%, most preferably at 100% amino acids selected from the group of autophagy-inhibiting amino acids alanine (A), glutamine (Q), leucine (L), valine (V) glycine (G) and proline (P). More preferably, the disclosure provides a stock solution, preferably aqueous, comprising a peptide-tartrate or a peptide citrate of a, preferably recombinant or synthetic, autophagy-inhibiting peptide, the peptide having an amino acid sequence comprising at least 50%, more preferably at least 75%, most preferably at 100% amino acids selected from the group of autophagy-inhibiting amino acids alanine (A), glutamine (Q), glycine (G), valine (V), leucine (L), and proline (P).

Heeding aggregation risk, a vial with a stock solution of AQGV (SEQ ID NO:2) peptide as defined here above for use in a clinical trial hitherto contained no more than (0.8 mol/L) active substrate in solution. Based on the current disclosure such a stock solution of an AQGV-salt (SEQ ID NO:2) of an organic acid, in particular, of AQGV (SEQ ID NO:2) peptide-maleate, AQGV (SEQ ID NO:2) peptide-acetate AQGV (SEQ ID NO:2) peptide-tartrate or AQGV (SEQ ID NO:2) peptide-citrate (but not of adenosine or adenosine monophosphate) now is provided having an amino acid sequence comprising at least 50%, more preferably at least 75%, most preferably at 100% amino acids selected from the group of autophagy-inhibiting amino acids alanine (A), glutamine (Q), glycine (G), valine (V), leucine (L) and proline (P), to contain at least 0.85 mol/L, more preferably at least 0.9 mol/L, more preferably at least 1 mol/L, more preferably at least 1.2 mol/L, more preferably at least 1.4 mol/L, more preferably at least 1.6 mol/L, most preferably at least 1.8 mol/L, of the AQGV (SEQ ID NO:2) peptide-acetate, AQGV (SEQ ID NO:2) peptide tartrate or AQGV (SEQ ID NO:2) peptide-citrate. In a more preferred embodiment, the disclosure provides a stock-solution of the AQGV (SEQ ID NO:2) peptide-tartrate or the AQGV (SEQ ID NO:2) peptide-citrate wherein the concentration of the AQGV (SEQ ID NO:2) peptide is in the range of 2 mol/L ( ) to 2.5 mol/L. In a more preferred embodiment, the disclosure provides a stock-solution of the AQGV-peptide-citrate wherein the concentration of the peptide-citrate is in the range of 2.5 mol/L to 3 mol/L. In a more preferred embodiment, the disclosure provides a stock-solution of the peptide-citrate wherein the concentration of the peptide-citrate is in the range of 3 mol/L to 3.5 mol/L. In a more preferred embodiment, the disclosure provides a stock-solution of the peptide-citrate wherein the concentration of the peptide-citrate is in the range of 3.5 mol/L to 4.5 mol/L. In a more preferred embodiment, the disclosure provides a stock-solution of the peptide-citrate wherein the concentration of the peptide-citrate is in the range of 4.5 mol/L to 5.5 mol/L. In a more preferred embodiment, the disclosure provides a stock-solution of the peptide-citrate wherein the concentration of the peptide citrate is equal to or larger than 5.5 mol/L. It is preferred that the stock solution is an aqueous solution.

It is preferred that the stock solution is an aqueous solution of autophagy-inhibiting amino acids comprising a dipeptide AQ, QQ, LQ, GQ, PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QLG, QGG, QPG, QVG, LAG, LGG, LPG, LVG or a tetrapeptide AQGV (SEQ ID NO:2), QQGV (SEQ ID NO:24), LQGV (SEQ ID NO:25), GQGV (SEQ ID NO:26), PQGV (SEQ ID NO:27), VQGV (SEQ ID NO:28), ALGV (SEQ ID NO:29), LLGV (SEQ ID NO:30), QLGV (SEQ ID NO:31), GLGV (SEQ ID NO:32), PLGV (SEQ ID NO:33), VLGV (SEQ ID NO:34), QAGV (SEQ ID NO:36), QLGV (SEQ ID NO:35), QGGV (SEQ ID NO:37), QPGV (SEQ ID NO:38), QVGV (SEQ ID NO:39), LAGV (SEQ ID NO:40), LGGV (SEQ ID NO:41), LPGV (SEQ ID NO:42), LVGV (SEQ ID NO:43), or a mixture thereof.

It is preferred that a peptide according to the disclosure has a peptide sequence with length of 2-40 amino acids, preferably 3-30 amino acids, preferably 4-20 amino acids. It is most preferred that the peptide according to the disclosure has a peptide sequence that comprises at least 6 amino acids, in particular, when at least 4 of those inhibit autophagy. A maximum length of a peptide-tartrate or peptide citrate according to the disclosure preferably comprises at most 50 amino acids, more preferably at most 40 amino acids, more preferably at most 30 amino acids, more preferably at most 20 amino acids, more preferably at most 15 amino acids, more preferably at most 12 amino acids, and most preferably at most 9 amino acids.

The disclosure provides a method for reducing p38 MAPK kinase activity leading to cytoskeleton reorganization, comprising providing cells, preferably having a formyl-peptide-receptor associated with their surface, with a source of autophagy-inhibiting amino acids, preferably wherein the source is an AQGV (SEQ ID NO:2) peptide as provided herein, the amino acids for least 50% selected from the group of alanine (in one letter code: A), glutamine (Q), glycine (G), valine (V), leucine (L), isoleucine (I), proline (P) and arginine (R).

The disclosure provides a method for reducing formyl-peptide-receptor (FPR)-mediated p38 MAPK kinase activity, comprising providing cells, preferably having a formyl-peptide-receptor associated with their surface, with a source of autophagy-inhibiting amino acids, preferably wherein the source is an AQGV (SEQ ID NO:2) peptide as provided herein, the amino acids for least 50% selected from the group of alanine (in one letter code: A), glutamine (Q), glycine (G), valine (V), leucine (L), isoleucine (I), proline (P) and arginine (R).

The disclosure provides a method for reducing PI3K/AKT/mTOR activity leading to cytoskeleton reorganization, comprising providing cells, preferably having a formyl-peptide-receptor associated with their surface, with a source of autophagy-inhibiting amino acids, preferably wherein the source is an AQGV (SEQ ID NO:2) peptide as provided herein, the amino acids for least 50% selected from the group of alanine (in one letter code: A), glutamine (Q), glycine (G), valine (V), leucine (L), isoleucine (I), proline (P) and arginine (R).

The disclosure provides a method for reducing formyl-peptide-receptor (FPR)-mediated PI3K/AKT/mTOR activity, comprising providing cells, preferably having a formyl-peptide-receptor associated with their surface, with a source of autophagy-inhibiting amino acids, preferably wherein the source is an AQGV (SEQ ID NO:2) peptide as provided herein, the amino acids for least 50% selected from the group of alanine (in one letter code: A), glutamine (Q), glycine (G), valine (V), leucine (L), isoleucine (I), proline (P) and arginine (R).

The disclosure provides a method for reducing cytoskeleton reorganization, comprising providing cells, preferably having a formyl-peptide-receptor associated with their surface, with a source of autophagy-inhibiting amino acids, preferably wherein the source is an AQGV (SEQ ID NO:2) peptide as provided herein, the amino acids for least 50% selected from the group of alanine (in one letter code: A), glutamine (Q), glycine (G), valine (V), leucine (L), isoleucine (I), proline (P) and arginine (R).

The disclosure provides a method for reducing formyl-peptide-receptor (FPR)-mediated cytoskeleton reorganization, comprising providing cells, preferably having a formyl-peptide-receptor associated with their surface, with a source of autophagy-inhibiting amino acids, preferably wherein the source is an AQGV (SEQ ID NO:2) peptide as provided herein, the amino acids for least 50% selected from the group of alanine (in one letter code: A), glutamine (Q), glycine (G), valine (V), leucine (L), isoleucine (I), proline (P) and arginine (R).

The disclosure provides a method for modifying vascular permeability comprising providing cells, preferably having a formyl-peptide-receptor associated with their surface, with a source of autophagy-inhibiting amino acids, preferably wherein the source is an AQGV (SEQ ID NO:2) peptide as provided herein, selected from the group of alanine (in one letter code: A), glutamine (Q), glycine (G), valine (V), leucine (L), isoleucine (I), proline (P) and arginine (R).

The disclosure provides a method for improving tissue repair comprising providing cells, preferably having a formyl-peptide-receptor associated with their surface, with a source of autophagy-inhibiting amino acids, preferably wherein the source is an AQGV (SEQ ID NO:2) peptide as provided herein, selected from the group of alanine (in one letter code: A), glutamine (Q), glycine (G), valine (V), leucine (L), isoleucine (I), proline (P) and arginine (R).

The disclosure provides a method according to the invention, wherein the peptide comprising the autophagy-inhibiting amino acids comprises a dipeptide AQ, QQ, LQ, GQ, PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QOLG, QGG, QPG, QVG, LAG, LGG, LPG, LVG or a tetrapeptide AQGV (SEQ ID NO:2), QQGV (SEQ ID NO:24), LQGV (SEQ ID NO:25), GQGV (SEQ ID NO:26), PQGV (SEQ ID NO:27), VQGV (SEQ ID NO:28), ALGV (SEQ ID NO:29), LLGV (SEQ ID NO:30), QLGV (SEQ ID NO:31), GLGV (SEQ ID NO:32), PLGV (SEQ ID NO:33), VLGV (SEQ ID NO:34), QAGV (SEQ ID NO:36), QLGV (SEQ ID NO:35), QGGV (SEQ ID NO:37), QPGV (SEQ ID NO:38), QVGV (SEQ ID NO:39), LAGV (SEQ ID NO:40), LGGV (SEQ ID NO:41), LPGV (SEQ ID NO:42), LVGV (SEQ ID NO:43), or a mixture thereof.

In another embodiment, this application finds the SARS-COV-2 spike protein (see also FIGS. 11-13) to carry a distinct peptide-motif sequence KWPWYIWL (SEQ ID NO:44) or variant KWPWYVWL (SEQ ID NO:45) in a membrane proximal external region (MPER) capable of binding to a receptor of the FPR-family of receptors. COVID-19 is activating FPR-mediated pathways, in particular, through interaction of vascular cells with its spike protein, therewith leading to vascular leakage, thrombotic events and modulating Ang1/Ang2 ratio. The MPER region may as well be involved in sparsely reported and incidental thrombotic events after vaccination with distinct coronavirus-based-vaccines that express the spike protein not fixated in a prefusion state. As AQGV (SEQ ID NO:2)-peptide inhibits formyl peptide-activated FPR-mediated pathways (p38-MK2-HSP27 and PI3K-AKT-mTOR, see FIG. 10) involved in disrupting vascular integrity, AQGV-peptide improves vascular leakage and thrombotic events by inhibiting thrombus formation and modulating Ang1/Ang2 ratio after events causing expression of at least the fusogenic region with motif KWPWYIWL (SEQ ID NO:44) or variant KWPWYVWL (SEQ ID NO:45) in a subject. Surprisingly, the fusogenic region with motif KWPWYIWL (SEQ ID NO:44) or variant KWPWYVWL (SEQ ID NO:45) also comprises an FPR-binding site participating in inducing vascular leakage in a subject. Therewith, the present application also provides alternative treatment or use. A method of treatment of a subject deemed to express a peptide or protein comprising a fusogenic region derived from a virus, the method comprising adoptive cell therapy using at least one cell provided with a receptor recognizing the fusogenic site. It is preferred that the fusogenic region at least comprises peptide motif KWPWYIWL (SEQ ID NO:44) or at least comprises peptide motif KWPWYVWL (SEQ ID NO:45), in particular, wherein the cell is a transformed T-cell, such as a CAR-T or TCR-T cell, preferably wherein the cell is directed against a (preferably CD8+) T-cell epitope comprising or overlapping the fusogenic region. Such adoptive cellular therapy (see, for example, June et al., Adoptive cellular therapy: A race to the finish line Science Translational Medicine 25 Mar. 2015: Vol. 7, Issue 280, pp. 280ps7) as here provided uses of at least one cell provided with a receptor recognizing a fusogenic region derived from a virus in method of treatment of a subject deemed to express a peptide or protein comprising the fusogenic region, wherein the fusogenic region at least comprises peptide motif KWPWYIWL (SEQ ID NO:44) or at least comprises peptide motif KWPWYVWL (SEQ ID NO:45), and wherein the cell is a transformed T-cell, such as a CAR-T or TCR-T cell preferably directed against a (preferably CD8+) T-cell epitope comprising or overlapping the fusogenic region.

### Further Embodiments

1. An AQGV (SEQ ID NO:2) peptide, or a functional analogue thereof, for use in the treatment of a human subject, the use comprising modifying hemodynamics in the human subject.

2. An AQGV (SEQ ID NO:2) peptide, or a functional analogue thereof, for use in the treatment of a human subject, wherein the human subject is suffering from a viral infection and wherein the use comprises modifying hemodynamics in the human subject.

3. An AQGV (SEQ ID NO:2) peptide, or a functional analogue thereof, for use in the treatment of a human subject having impaired lung function, the use comprising modifying hemodynamics and improving hypoxemia in the human subject.

4. An AQGV (SEQ ID NO:2) peptide, or a functional analogue thereof, for use as in accordance with any one of further embodiments 1-3, wherein the use reduces fluid retention in the human subject.

5. An AQGV (SEQ ID NO:2) peptide, or a functional analogue thereof, for use in accordance with any of further embodiments 1-4 wherein the use comprises a reduced use of vasopressive agents.

6. An AQGV (SEQ ID NO:2) peptide, or a functional analogue thereof, for use in accordance with any one of further embodiments 1-5 wherein the use comprises a reduced fluid intake.

7. An AQGV (SEQ ID NO:2) peptide, or a functional analogue thereof, for use in accordance with further embodiment 5, wherein the reduced use of vasopressive agents comprises a reduced duration of vasopressive agent use.

8. An AQGV (SEQ ID NO:2) peptide, or a functional analogue thereof, for use in accordance with any one of further embodiments 3-6, wherein the subject is suffering from a respiratory viral infection.

9. An AQGV (SEQ ID NO:2) peptide, or a functional analogue thereof, for use in accordance with any one of further embodiments 3-8 wherein the use improves lung function in the human subject.

10. An AQGV (SEQ ID NO:2) peptide, or a functional analogue thereof, for use in accordance with further embodiment 9, wherein the improved lung function involves an improved oxygen saturation of blood.

11. An AQGV (SEQ ID NO:2) peptide, or a functional analogue thereof, for use in accordance with any one of further embodiments 3-10, wherein the human subject has impaired lung function the impaired lung function being ARDS.

12. An AQGV (SEQ ID NO:2) peptide, or a functional analogue thereof, for use as in accordance with any one of further embodiments 1-11, wherein the use reduces leakage of plasma from the blood to peripheral tissue and/or organs.

13. An AQGV (SEQ ID NO:2) peptide, or a functional analogue thereof, for use in accordance with any one of further embodiments 1-12, wherein the use is in a human subject suffering from or at risk of suffering from detrimental effects of mechanical ventilation.

14. An AQGV (SEQ ID NO:2) peptide, or a functional analogue thereof, for use in accordance with any one of further embodiments 1-13, wherein the use is in a human subject at risk of having edema.

15. An AQGV (SEQ ID NO:2) peptide, or a functional analogue thereof, for use in accordance with any one of further embodiments 2-14, wherein the human subject is suffering or thought to be suffering from to corona virus infection.

16. An AQGV (SEQ ID NO:2) peptide, or a functional analogue thereof, for use in accordance with further embodiment 15, wherein the infection is SARS-Cov-2 infection.

17. An AQGV peptide, or a functional analogue thereof, for use in accordance with any one of further embodiments 1-16, wherein the peptide is administered into the bloodstream.

18. An AQGV (SEQ ID NO:2) peptide, or a functional analogue thereof, for use in accordance with further embodiment 17, wherein the peptide is administered at a rate of at least 70 mg/kg body weight/hour.

19. An AQGV (SEQ ID NO:2) peptide, a functional analogue thereof, for use in accordance with further embodiment 15 or further embodiment 16, wherein the peptide is administered for at least 1 hour.

20. An AQGV (SEQ ID NO:2) peptide, a functional analogue thereof, for use in accordance with any one of further embodiments 15-19, wherein the administration is intermittent.

21. An AQGV (SEQ ID NO:2) peptide, or a functional analogue thereof, for use in accordance with any one of further embodiments 1-20, wherein the human subject is admitted into intensive care, and wherein the use improves parameters measured of the human subject, the parameters of the human subject determined to assess remaining in intensive care.

22. An AQGV (SEQ ID NO:2) peptide, or a functional analogue thereof, for use in accordance with further embodiment 21, wherein the improvement in parameters results in a reduced length of stay at intensive care.

23. An AQGV (SEQ ID NO:2) peptide, or a functional analogue thereof, for use as in accordance with any one of further embodiments 1-22, wherein the uses induces vasoconstriction.

24. An AQGV (SEQ ID NO:2) peptide, or a functional analogue thereof, for use as in accordance with any one of further embodiments 1-23, wherein the subject is deemed at risk of VALI or VILI.

25. An AQGV (SEQ ID NO:2) peptide, or a functional analogue thereof, for use as in accordance with any one of further embodiments 1-23, wherein the subject is deemed to express a peptide or protein comprising a fusogenic region derived from a virus.

26. An AQGV (SEQ ID NO:2) peptide, or a functional analogue thereof, for use as in accordance with further embodiment 25, wherein the fusogenic region at least comprises peptide motif KWPWYIWL (SEQ ID NO:44) or variant KWPWYVWL (SEQ ID NO:45).

27. An AQGV (SEQ ID NO:2) peptide, or a functional analogue thereof, for use as in accordance with further embodiment 25 or 26, wherein the fusogenic region at least comprises an FPR-binding site.

28. A method of treatment comprising administering an AQGV (SEQ ID NO:2) peptide, or a functional analogue thereof, to a human subject, the human subject being in need of maintaining hemodynamic stability.

29. A method of treatment comprising administering an AQGV (SEQ ID NO:2) peptide, or a functional analogue thereof, to a human subject, the human subject being in need of improving hemodynamic stability.

30. A method of treatment comprising administering an AQGV (SEQ ID NO:2) peptide, or a functional analogue thereof, to a human subject, the human subject having impaired lung function, wherein the treatment of administering an AQGV (SEQ ID NO:2) peptide comprises maintaining or improving hemodynamic stability in the human subject.

31. A method of treatment comprising administering an AQGV (SEQ ID NO:2) peptide, or a functional analogue thereof, intermittently to a human subject, the human subject having impaired lung function, wherein the treatment of administering an AQGV (SEQ ID NO:2) peptide comprises maintaining or improving hemodynamic stability in the human subject.

31. A method of treatment of a subject deemed to express a peptide or protein comprising a fusogenic region derived from a virus, the method comprising adoptive cell therapy using at least one cell provided with a receptor recognizing the fusogenic site.

32. A method according to further embodiment 31 wherein the fusogenic region at least comprises peptide motif KWPWYIWL (SEQ ID NO:44).

33. A method according to further embodiment 31 wherein the fusogenic region at least comprises peptide motif KWPWYVWL (SEQ ID NO:45).

33. A method according to any of further embodiments 31 to 33 wherein the cell is a transformed T-cell, such as a CAR-T or TCR-T cell.

34. A method according to embodiment 33 wherein the cell is directed against a T-cell epitope comprising or overlapping the fusogenic region.

35. Use of at least one cell provided with a receptor recognizing a fusogenic region derived from a virus in method of treatment of a subject deemed to express a peptide or protein comprising the fusogenic region.

36. Use according to further embodiment 35 wherein the fusogenic region at least comprises peptide motif KWPWYIWL (SEQ ID NO:44).

37. Use according to further embodiment 35 wherein the fusogenic region at least comprises peptide motif KWPWYVWL (SEQ ID NO:45).

38. Use according to any of further embodiments 35 to 37 wherein the cell is a transformed T-cell, such as a CAR-T or TCR-T cell.

39. Use according to embodiment 38 wherein the cell is directed against T-cell epitope comprising or overlapping the fusogenic region.

### BRIEF DESCRIPTION OF THE DRAWINGS

FIG. 1: Early infections with the SARS-Cov-2 virus mostly run a mild or even uneventful course. That Stage I course is seen in >80% of people infected. This majority of patients experience an upper airway infection of nose and throat, with a dry cough, that generally passes in 2-12 days. In the remaining <20% of cases, two distinct pathological stages may develop, often starting at around the time that in mild cases the viral infection is to reduce due to an emerging immune response directed against the virus. Stage II is a viral pneumonia (pulmonary phase with lung injury) with permeability losses of the alveolar-capillary membrane (Stage IIA) that diffusely and profusely affects the deeper airways and alveoli of both lungs (Stage IIB), causing reduced uptake of oxygen due to respiratory failure. This two-sided pneumonia may be followed by stage III, a full-blown disease with general malaise, high fever and ultimately organ (kidney, liver, heart) failure at large.

FIG. 2: Acute disease in Stage II increases vascular permeability and results in fluid leakage from lung capillaries into the lung tissues (see also lung injury in FIG. 3). This angiopoietin regulated permeability is depicted here. Angiopoietin 1 (ANG1) is constitutively secreted by perivascular mural cells. When gaps form between cells, ANG1 is released in the vascular lumen. Ligand-binding of (ANG1) to TIE2 induces sequestration of the tyrosine kinase Src and thus establishes stable expression of VE-cadherin on the surface of the endothelial cell, allowing gaps to close. ANG2 is stored in Weibel-Palade (WBP) bodies and rapidly released upon triggering signals. Its binding to TIE2 abolishes ANG1-induced sequestration of Src, resulting in the internalization of VE-cadherin.

FIG. 3: Acute disease in stage II results in lung injury characterized by edematous lung tissues causing low gas (oxygen and carbon-dioxide) diffusing capacity. SARS-COV-2 infection starts in Type II cells. The permeability of lung-capillaries is increased by increased vascular cell gap formation as depicted in FIG. 2. This process generates a flux of fluid from the capillaries into the thin layer of extra-cellular-matrix separating alveoli from capillaries, with intercellular fluid retention therewith thickening (swelling) the membrane through accumulation of fluid in the extra-cellular-matrix (causing a widened edematous interstitium), separating alveolar cells from vascular cells, and entering the alveoli. This typically evokes local inflammatory activity of white blood cells that migrated to the lung tissue. All-in-all, gas diffusion is severely hampered causing difficulties breathing. In such Stage II lung injury patients, plasma levels of biomarkers of endothelial activation, as may be measured by ELISA, are often predictive of mortality. In particular, the concentration of angiopoietin-2 relative to angiopoietin-1 (Ang-2/Ang-1) may be a useful biologic marker of mortality in acute lung injury (ALI) patients.

FIG. 4: Schematic depiction in the infection stages of FIG. 1 of intermittent dosing of AQGV (SEQ ID NO:2)-peptide in various stages of COVID-19. Shadowed bars indicate time slots wherein AQGV (SEQ ID NO:2) peptide or related substance is administered intravenously, preferably at a rate of at least 75 mg/kg/hour, more preferably at least 90 mg/kg/hour. It is, in particular, useful to administer the AQGV (SEQ ID NO:2) peptide or related substance intermittently. Preferred use is dosing for 2-4 hours of at least 75 mg/kg/hour, more preferably at least 90 mg/kg/hour, then optionally reducing to 30 mg/kg/hour for 2-4 hours (in between shaded bars), or for as long as it takes to monitor the patients response to treatment by clinical or laboratory diagnosis, or stop administering the substance for 1-2 hours until diagnostic studies have completed, and then resume treatment with 2-4 hours of at least 75 mg/kg/hour, more preferably at least 90 mg/kg/hour. Depending on the stage of disease, therapeutic effects of EA-230 may be monitored by determining hypoxia, plasma Ang2/Ang1 ratio and plasma levels of D-dimer in Stage II and III.

FIG. 5: The need for treatment of hemodynamic instability by use of vasopressors (left) and by use of fluid therapy to adjust net fluid balance were considerably improved in the first 24 hours of intensive care unit (ICU) in those patients given EA-230 peptide (SEQ ID NO:2). Therewith, EA-230 (SEQ ID NO:2) significantly improves hemodynamic recovery, providing a significant improvement of hemodynamic stability (reducing a composite measure of required fluid therapy and blood pressure medication; 2-way ANOVA; p=0.006).

FIG. 6: Both the ICU (p=0.02) and hospital (p=0.001) length-of-stay was shorter in patients treated with EA-230 (AQGV (SEQ ID NO:2)) compared to the placebo group. AQGV (SEQ ID NO:2)-peptide EA-230 reduced the number of patients at the ICU at 24 hours by 48%; and reduced hospital length of stay by 20%.

FIG. 7: Overview of solubility experiments with results in Table 1.

FIG. 8: Based on the results depicted in FIG. 7 the concentration below which an aggregated peptide-salt tends to resolve of the neutral-peptides salts screened were determined (aggregation points). It can be concluded that changing the anion significantly influences the solubility characteristics of AQGV (SEQ ID NO:2). Higher solubility (solubility in 0.9% NaCl) and therewith higher aggregation points were observed for the AQGV (SEQ ID NO:2)-citric acid (AQGV-citrate) and -tartaric acid (AQGV-tartrate (SEQ ID NO:2)) salt, whereas maleic acid and KHSO4 salts showed lower solubility, compared to AQGV-Ac (SEQ ID NO:2). Using adenosine-monophosphate or adenosine did not provide solubility. Citric acid seems to be a special case. Highly concentrated solution does not crystallize or aggregate but tend to form a highly viscous solution.

FIG. 9: Formyl-peptide-receptor-mediated vascular permeability after cell and tissue trauma. The human formyl peptide receptor (FPR) is N-glycosylated and activates cells via G(i)-proteins. Site-directed mutagenesis of extracellular Asn residues prevented FPR glycosylation but not FPR expression in cell membranes. However, in terms of high-affinity agonist binding, kinetics of GTPgammaS binding, number of G(i)-proteins activated, and constitutive activity, non-glycosylated FPR is much less active than native FPR. Mitochondrial N-formyl peptides (F-MIT) released from trauma/cell damage activate formyl peptide receptor (FPR) leading to changes in endothelial cell cytoskeleton, which subsequently induces endothelial contraction and vascular permeability, leukocyte extravasation and hypotension. N-Formyl peptides are common molecular signatures of bacteria and mitochondria that activate the formyl peptide receptor (FPR). FPR activation by mitochondrial N-formyl peptides (F-MIT) elicits changes in cytoskeleton-regulating proteins in endothelial cells that lead to increased endothelial cell contractility with increased vascular leakage and extravasation of leukocytes. FPR activation via mitochondrial N-formyl peptides (F-MIT) originating from tissue damage after injury such as trauma is a key contributor to impaired barrier function following cell and tissue injury or trauma, resulting in detrimental vascular effects such as adverse vascular permeability with edema, vascular leakage, adverse leukocyte extravasation and hypotension.

FIG. 10: Formyl-peptide-receptor-mediated peptide effects. FPR-activation of FPR-expressing cells with prototype FPR-ligand fMLP causes rapidly induced and significant (p<0.05; p38 from 60 to 600 sec, PKB at 600 sec) changes in phosphorylation status of PKB (also known as AKT) (FIG. 10a) and p38 MAPK kinases (FIG. 10c), but not (or not detected) in STAT3, JNK (FIG. 10b) and P42/p44MAPK/ERK1,2 (FIG. 10d) kinases. AQGV peptide effects on p38 MAPK (FIG. 10c) are already detected at 30 seconds after FPR-stimulation, AQGV peptide effects on PKB(AKT) follow (FIG. 10a) in a bi-phasic pattern at 300 sec. Both AQGV peptide effects on p38 and PKB-mediated signaling last for the full 600 seconds tested whereas the other kinases tested were not affected throughout. This acute and specific response to treatment shows specific and rapid effects of autophagy-inhibiting-AQGV (SEQ ID NO:2) peptide on p38 signaling in the context of regulation of the PI3K/AKT/mTOR pathway. The pathway is governing the balance between proteolysis and proteogenesis regulating cytoskeleton changes affecting vascular permeability. It is shown that AQGV (SEQ ID NO:2) peptide reduces p38 MAPK kinase activated changes as well as reduces PI3K/AKT/mTOR activated induced changes in cell cytoskeleton reorganization affecting endothelial cell contraction and adverse vascular permeability. AQGV (SEQ ID NO:2) peptide is useful and capable of addressing adverse vascular permeability, such as manifested by edema with vascular leakage, adverse leukocyte extravasation and hypotension in human subjects.

FIG. 11: AQGV (SEQ ID NO:2)-peptide to target viral-spike-protein-induced pulmonary and vascular leakage in Coronavirus infections as seen in SARS, MERS and COVID-19. SARS-CoV-2 spike (S) glycoproteins (SEQ ID NO: 80-SEQ ID NO: 86) are class I viral fusion proteins that promote viral entry into cells and are the main target of antibodies (White et al., Critical reviews in biochemistry and molecular biology. 2008 Jan. 1; 43(3):189-219). The C terminal end of spike protein contains a heptad repeat (HR2), a short linker region (the membrane proximal external region or MPER), a transmembrane helix domain (TMD) and a C-terminal cytoplasmic or internal domain (CTD/IC). After binding of the ACE2 receptor on the target cell to the receptor binding domain (RBD) on S protein, the heptad repeat 1 (HR1) and heptad repeat 2 (HR2) domains form a six-helix bundle fusion core (6HB), bringing the viral with the fusogenic MPER domain and cellular membranes together for fusion and cell entry (Walls et al., Tectonic conformational changes of a coronavirus spike glycoprotein promote membrane fusion. Proceedings of the National Academy of Sciences. 2017 Oct. 17; 114(42):11157-62; Xia et al., Fusion mechanism of 2019-nCoV and fusion inhibitors targeting HR1 domain in spike protein. Cellular & molecular immunology. 2020 Feb. 11:1-3). MPER is essential for viral entry into cells as identified in FIG. 12. Note that at least one T cell epitope, allowing generation of CD8+ T-cell cross-reactivity against SARS-CoV-2 and other coronavirus strains (Lee et al, Front. Immunol., 5 Nov. 2020 https://doi.org/10.3389/fimmu.2020.579480) overlaps with the fusogenic site as identified in FIG. 12. In patients, virus-specific CD4⁺ and CD8⁺ T cell responses are associated with milder disease, suggesting an involvement of the fusogenic region in protective immunity against COVID-19. Typically, the fusogenic site, and therewith the T-cell epitope, is strongly conserved in SARS-COV-2 (Guo E., Guo H. (2020) CD8 T cell epitope generation toward the continually mutating SARS-CoV-2 spike protein in genetically diverse human population: Implications for disease control and prevention. PLOS ONE 15(12): e0239566), herein it is provided to develop adoptive cellular therapy (ACT) directed against the fusogenic region that may be used in viral or vaccine based infections such as with corona virus or vaccine.

FIG. 12: The short membrane proximal external region (MPER) connects the HR2 and transmembrane domain, and contains an aromatic-amino-acid-rich fusogenic peptide sequence that destabilizes the membrane during fusion (Mahajan M., Bhattacharjya S. NMR structures and localization of the potential fusion peptides and the pre-transmembrane region of SARS-CoV: Implications in membrane fusion. Biochimica et Biophysica Acta (BBA)-Biomembranes. 2015 Feb. 1; 1848(2):721-30; Guillén J., Kinnunen P. K., Villalain J. Membrane insertion of the three main membranotropic sequences from SARS-CoV S2 glycoprotein. Biochimica et Biophysica Acta (BBA)-Biomembranes. 2008 Dec. 1; 1778(12):2765-74). This fusogenic region may sometimes be referred to as “membrane proximal ectodomain region” or “pre-transmembrane region” (PTM). The MPER peptides 1185-LGKYEQYIKWPWYVWLGF-1202 (SEQ ID NO:87) and 1193-KWPWYVWLGFIAGLIAIV-1210 (SEQ ID NO:88) from SARS-CoV-1 have been shown to intercalate into lipid membranes and to be highly surface active; the corresponding fusogenic sequences in SARS-CoV-2 and MERS-CoV are identical except for a V to I substitution at position 1216 (SEQ ID NO:89-SEQ ID NO:91).

FIG. 13: Alignment of 36-mer AA sequences of near-C-terminal (position 1183-1218 in SARS-COV-2) with coronavirus spike proteins that have shown binding to FPR-receptor (with increased binding at polymorphism W190/N192). Synthesized 11/12-meric peptides having FPR-binding activity shown in red (SEQ ID NO:92-SEQ ID NO:97). This application finds the SARS-COV-2 spike protein to carry a distinct and conserved fusogenic motif in its MPER domain (KWPWYIWL (SEQ ID NO:44)) that is capable of binding to FPR. This motif is highly homologous to related coronavirus spike protein motifs for which binding to FPR has been demonstrated. (Mills, Biochim. Biophys. Acta Mol. Basis Dis. 2006 July; 1762(7): 693-704). Vascular leakage in COVID-19 is at least partly modulated by binding and/or fusing of this spike protein comprising at least the minimally essential fusogenic sequence KWPWYIWL (SEQ ID NO:44) or variant KWPWYVWL (SEQ ID NO:45) to pulmonary vascular cells carrying the formyl-peptide receptor, and therewith may cause thrombotic events in coronavirus infection or vaccination against corona with a spike protein-vaccine such as ChAdOx1-S, in particular, when such a vaccine is not modified to express the spike proteins in a prefusion state only. FPR-mediated pathways are known to be activated in thrombotic events (Salamah et al., The formyl peptide fMLF primes platelet activation and augments thrombus formation. J. Thromb. Haemost. 2019; 17: 1120-1133) as well as in an acute lung injury and acute respiratory disease syndromes (ALI/ARDS) with pulmonary vascular leakage as a major clinical symptom (Thorax. 2017; 72:928-936). COVID-19 (in particular, the severe) infections with SARS-COV-2 typically increase risks on ALI/ARDS with pulmonary vascular leakage, leading to major fatalities.

### DETAILED DESCRIPTION

### Examples

Autophagy-Inhibiting Peptides

One Letter Code

In describing protein or peptide composition, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophane residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

Peptides

“Peptide” shall mean herein a natural biological or artificially manufactured (synthetic) short chain of amino acid monomers linked by peptide (amide) bonds. Glutamine peptide shall mean herein a natural biological or artificially manufactured (synthetic) short chain of amino acid monomers linked by peptide (amide) bonds wherein one of the amino acid monomers is a glutamine. Chemically synthesized peptides generally have free N- and C-termini. N-terminal acetylation and C-terminal amidation reduce the overall charge of a peptide; therefore, its overall solubility might decrease. However, the stability of the peptide could also be increased because the terminal acetylation/amidation generates a closer mimic of the native protein. These modifications might increase the biological activity of a peptide and are herein also provided.

Peptide Synthesis

In this application, peptides are either synthesized by classically known chemical synthesis on a solid support (Ansynth BV, Roosendaal, The Netherlands) or in solution (Syncom BV, Groningen, The Netherlands and Diosynth BV, Oss, The Netherlands). Pharmaceutical peptide compositions may be synthesized using trifluoroacetate as a counter-ion or salt after which trifluoroacetate is exchanged by a counter-ion such as maleate (from maliec acid), acetate (from acetic acid), tartrate (from tartaric acid) or citrate (from citric acid). The drug substance of AQGV (EA-230) for use in pre-clinical and clinical human studies has been manufactured by Organon N.V (formerly Diosynth B.V.), (Oss, The Netherlands), whereas filling and finishing of the final product has been performed by Octoplus Development, Leiden (The Netherlands). Molecular weight of EA-230 (AQGV) is 373 g/mol).

FPR-Mediated Vascular Permeability and Hypotension

Although the concept that active contraction of endothelial cells regulate vascular permeability was first suggested by Majno in 1961 (J. Biophys. Biochem. ytol (1961) 11:571.10.1083/jcb.11.3.571), currently the intracellular events regulating endothelial contractile activity are still relatively unknown. N-Formyl peptides are common molecular signatures of bacteria and mitochondria that activate the formyl peptide receptor (FPR). FPR activation by mitochondrial N-formyl peptides (F-MIT) or by bacterial N-formyl peptides (F-MLP) such as N-formyl-methionyl-leucyl-phenylalanine elicits changes in cytoskeleton-regulating proteins in endothelial cells that lead to increased endothelial cell contractility with increased vascular leakage and extravasation of leukocytes. FPR activation is a key contributor to impaired barrier function in following trauma. It has been proposed that in patients, mitochondrial components from damaged tissue can initiate the genesis of vascular leakage (Wenceslau et al., Front Immunol. 2016; 7: 297). For evolutionary reasons, mitochondria share several characteristics with bacteria, and when fragments of mitochondria are released into the circulation, they are recognized by cells carrying the formyl-peptide-receptor (FPR). Due to protein translation initiation by formyl-methionine in both bacteria and mitochondria, N-formyl peptides are common molecular signatures of bacteria and mitochondria and are known to play a role in the initiation of vascular leakage by activating the formyl peptide receptor (FPR).

The vasculature, composed of vessels of different morphology and function, distributes blood to all tissues and maintains physiological tissue homeostasis. Among others, to illustrate its central role in maintaining homeostasis, the vasculature not only serves as the main carrier in gas exchange from lung to tissues (e.g., oxygen (and vice versa, e.g., carbon dioxide)) but also carries nutrients from gut to liver to tissues and toxic by-products resulting metabolism from tissues to kidney to urine for excretion.

In a range of pathologies, the vasculature is often affected by, and engaged in, the disease process. This foremost results in adverse vascular permeability with edema, adverse vascular leakage, adverse leukocyte extravasation and hypotension and may also result in excessive formation of new, unstable, and hyper permeable vessels with poor blood flow, which further promotes hypoxia and disease propagation. Chronic adverse vessel permeability may also facilitate metastatic spread of cancer. Thus, there is a strong incentive to learn more about (and be able to modulate) an important aspect of vessel biology in health and disease: the regulation of vessel permeability.

Endothelial cells in different vessels and in different organs have distinct functions and morphologies (Aird W. C., Molecular heterogeneity of tumor endothelium. Cell Tissue Res. 2009; 335:271-81), but in general serve to provide a barrier between blood and tissue. In certain organs, such as the brain and in endocrine organs, endothelial cells present certain morphological features that reflect the need for communication between the organs and the circulation. In the brain, the vasculature forms a particularly strong barrier, the blood-brain barrier (BBB) to protect the brain parenchyma from detrimental edema. In hormone-producing organs, such as the endocrine pancreas, endothelial cells display specialized fenestrae on their surface. These are diaphragm-covered “holes” in the plasma membrane, which allow extremely rapid exocytosis of hormones. In most organs, the endothelial cells form a dynamic barrier between the blood and the tissue. In resting conditions, the vasculature continuously leaks solute and small molecules but restricts extravasation of larger molecules and cells. In many diseases, including cancer, the vascular barrier disintegrates and leakage increases and may become chronic. The leakage of larger molecules and cells may result in edema, adverse leukocyte extravasation and hypotension, and often disease progression.

It is well recognized that, for example, kinins such as bradykinin are involved in a series of physiological and sometimes pathological vascular responses affecting endothelial barrier function. Most of their actions are mediated by the activation of 2 G protein-coupled receptors, named B1 and B2. The activation of kinin receptors may play a key role in the modulation of atherosclerotic risk through the promotion of microangiogenesis, inhibition of vascular smooth muscle cell growth, coronary vasodilatation, increased local nitric oxide synthesis, or by exerting antithrombotic actions. The bradykinin B1 receptor (B1R) is typically absent under physiological conditions, but is highly inducible following tissue injury, stress, burns, traumatic damage, such as, for example, recently reported in COVID-19 disease.

Damage induced by tissue injury may cause a significant and time-dependent increase in des-Arg9-bradykinin (des-Arg9-BK) responsiveness that parallels B1R mRNA expression. It induces the activation of some members of the mitogen activated protein kinase (MAPK) family, namely, extracellular signal-regulated kinase (ERK) and p38 MAPK. The blockade of p38 MAPK but not ERK pathways with selective inhibitors, results in a significant reduction of the upregulated contractile response caused by the selective B1R agonist des-Arg9-BK, and largely prevents the induction of B1R mRNA expression enhancing tissue damage induced adverse vascular permeability.

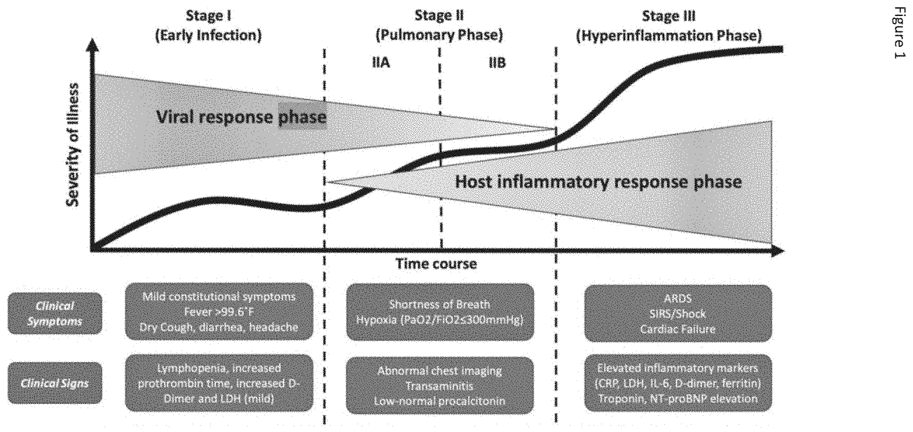

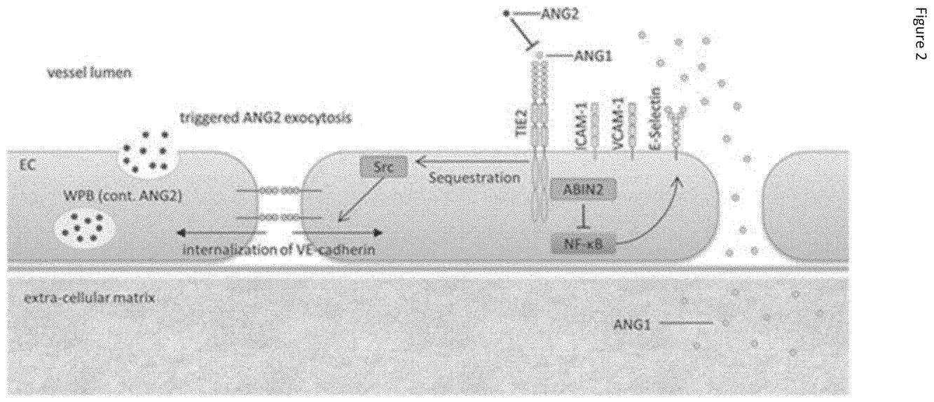

Among other stress stimuli, exposure to hypoxia as a consequence of impaired blood flow, or as a consequence of impaired gas exchange between alveoli and the surrounding capillaries, also causes structural changes in the endothelial cell layer of blood vessels that alter its permeability and its interaction with leukocytes and platelets. These structural changes again cause impaired endothelial cell barrier function resulting in detrimental vascular effects such as adverse vascular permeability with edema, vascular leakage, adverse leukocyte extravasation and hypotension (see also FIG. 1), and may further deteriorate gas exchange from lung to blood and from blood to tissue, and vice versa.

One of the well characterized cytoskeletal changes in response to stress involves the reorganization of the actin cytoskeleton and the formation of stress fibers. Kayyali et al. (J. Biol. Chem. (2002) 277(45): 42596-602) describe cytoskeletal changes in pulmonary microvascular endothelial cells in response to hypoxia and potential mechanisms involved in this process. The hypoxia-induced actin redistribution appears to be mediated by components downstream of MAPK p38, which is activated in pulmonary endothelial cells in response to hypoxia. Results indicate that kinase MK2, which is a substrate of p38, becomes activated by hypoxia, leading to the phosphorylation of one of its substrates, HSP27. As another example, F-actin rearrangement is also an early event in burn-induced endothelial barrier dysfunction, and HSP27, a target of p38 MAPK/MK2 pathway, plays an important role in actin dynamics. As HSP27 phosphorylation is known to alter actin distribution and thus contractility of cells, Kayyali et al., provide that the p38-MK2-HSP27 pathway causes changes in vascular permeability due to actin redistribution, as, for example, observed in hypoxia.

Taken together, these results indicate that tissue damage stimulates the p38-MK2-HSP27 pathway leading to significant alteration in the actin cytoskeleton. It has previously also been shown that inhibition of the p38 MAPK pathway ameliorates vascular dysfunction by significantly reducing endothelial cell contraction (Wang et al., APMIS (2014) 122(9):832).

In recent years, another pathway, the PI3K/AKT/mTOR [phosphatidylinositol 3′ kinase (PI3K), protein kinase B (PKB or AKT) and mammalian target of rapamycin (mTOR)] pathway, has also been identified to be essential for regulating endothelial cell contractility and Tsuji-Tamura and Ogawa indeed (Journal of Cell Science 2016 129: 1165-1178) identified inhibitors of phosphatidylinositol 3-kinase (PI3K)-Akt-pathway and inhibitors of mammalian target of rapamycin complex 1 (mTORC1) inhibitors as potent inducers of endothelial cell elongation required for restoring vascular permeability governed by vascular endothelial cells. Such elongation is required to fill the gaps that form between endothelial cells when these cells contract after p38-MK2-HSP27 and/or PI3K/AKT/mTOR signaled cytoskeleton reorganization. It is these gaps (again see FIG. 1) through which adverse leakage and adverse extravasation occurs that explains the resulting edema, vascular leakage, adverse leukocyte extravasation and loss of vascular fluid with a risk for hypotension.

Closing of these gaps is in general governed by the ratio of various angiogenic factors such as angiopoietin-2 to angiopoietin-1 at the site of increased vascular permeability, whereby angiopoetin-2 in general induces endothelial cell apoptosis (there with enhancing gap-formation) and angiopoietin-1 counters gap formation by facilitating endothelial vcel elongation and gap closure. Inhibition of the p38 pathway, but not of the ERK1/2 pathway, attenuates angiopoetin-2-mediated endothelial cell apoptosis (Li et al., Exp. Ther. Med. 2018 December; 16(6): 4729-4736. Published online 2018 Oct. 1)). In addition, the PI3K/AKT/mTOR pathway modulates the expression of other angiogenic factors as nitric oxide and angiopoietins (Karar and Mayti, Front. Mol. Neurosci., 2 Dec. 2011, https://doi.org/10.3389/fnmo1.2011.00051).

Thus, inhibiting signaling events in the p38/p38-MK2-HSP27 and/or PI3K/AKT/mTOR pathways—that signal cytoskeleton contraction—reduces vascular permeability, and therewith reduces adverse permeability and adverse extravasation, with resulting edema, vascular leakage, adverse leukocyte extravasation and loss of vascular fluid with a risk of hypotension. Methods and means for such inhibition are objects of this disclosure.

Use of EA-230 in Mitigating Ventilation Requirements and Ventilation-Associated-Lung-Injury in COVID-19

Infections with the SARS-Cov-2 virus that causes COVID-19 mostly run a mild or even uneventful course. That course is seen in >80% of people infected. This majority of patients experience an upper airway infection of nose and throat, with a dry cough, that generally passes in 2-12 days, after which the virus will have gone from the body. These patients may or may not experience common flu-like signs such as fever, fatigue, headache and muscle pain during the period of infection with the virus. They may not need treatment with AQGV (SEQ ID NO:2)-peptide according to the invention.

In the remaining cases distinct pathological states may develop (FIGS. 1, 2 and 3), often starting at around the time that in mild cases the viral infection is considered bound to reduce due to an emerging immune response directed against the virus. First a viral pneumonia appears with increased vascular permeability in the lungs (pulmonary phase) that diffusely and profusely affects the deeper airways and alveoli of both lungs, causing reduced uptake of oxygen and respiratory failure. This two-sided pneumonia may be rapidly followed by full-blown systemic disease with general malaise, high fever and ultimately organ (kidney, liver, heart) failure. Patients that show these symptoms and have difficulties breathing will typically be admitted to a hospital, may enter the intensive care unit (ICU), may be put on a ventilator and need to be put into an induced coma and, even then, in its worse course, may die. These patients may very well be helped with treatment with AQGV (SEQ ID NO:2)-peptide.

Increased Vascular Permeability Leads to Respiratory Failure.

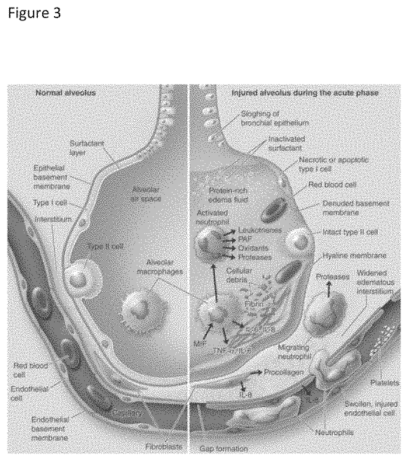

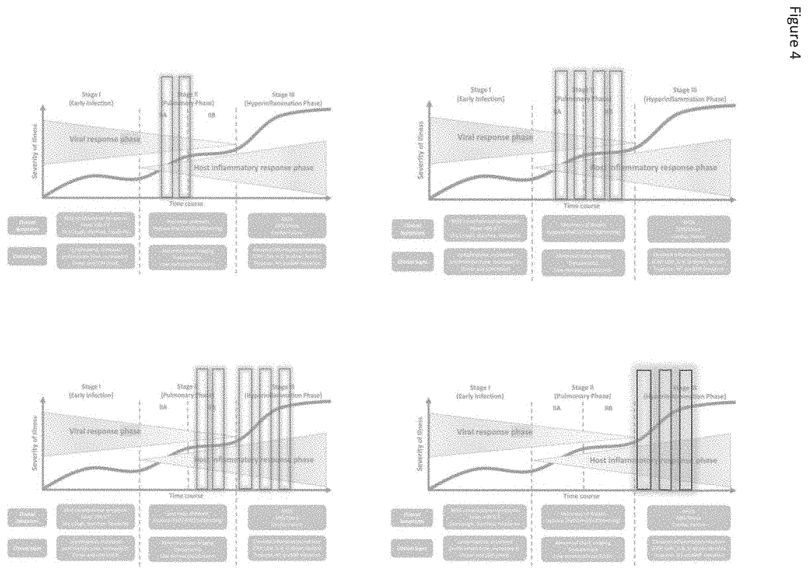

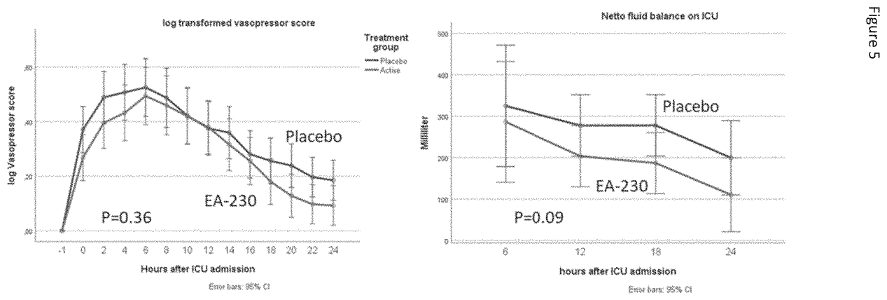

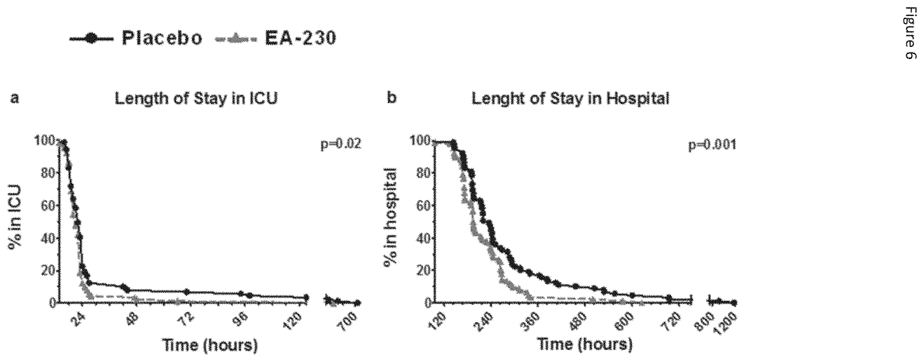

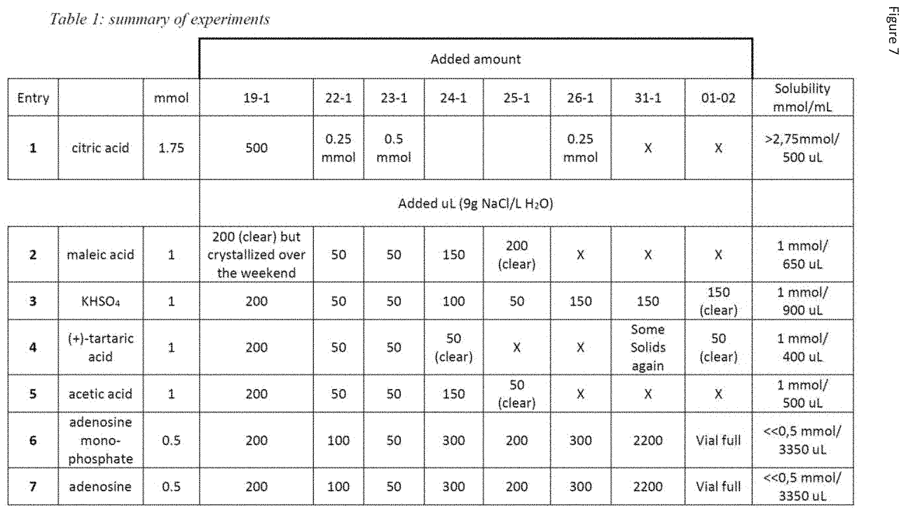

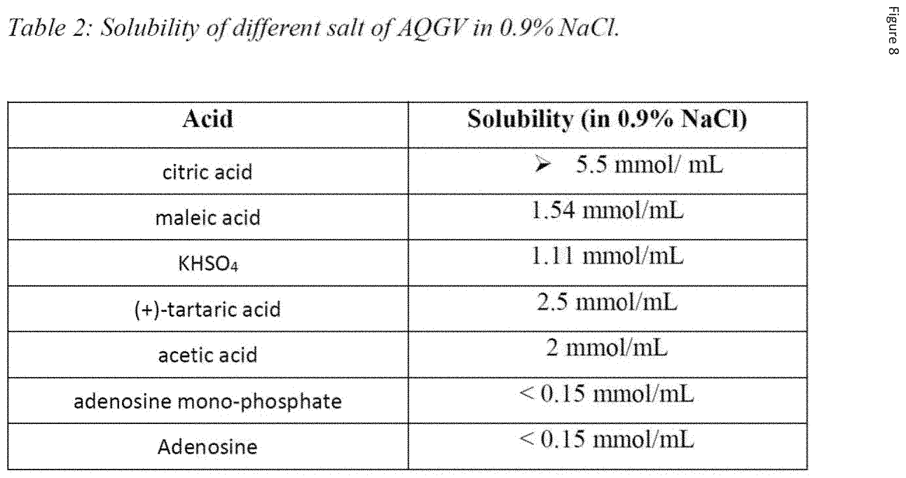

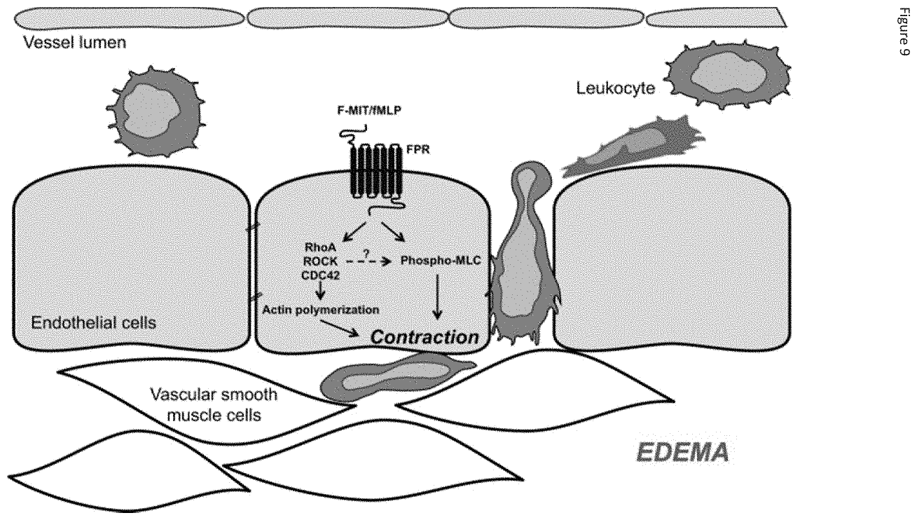

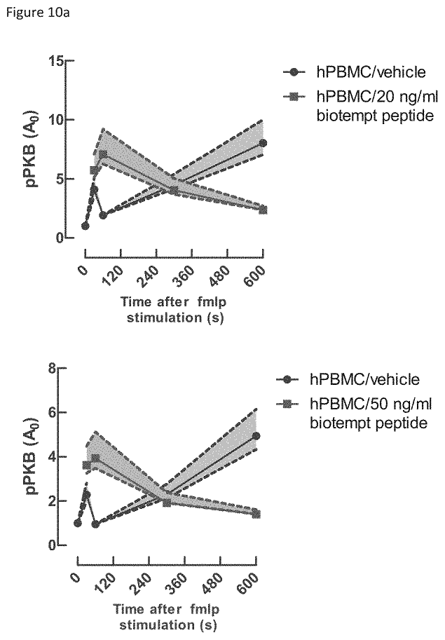

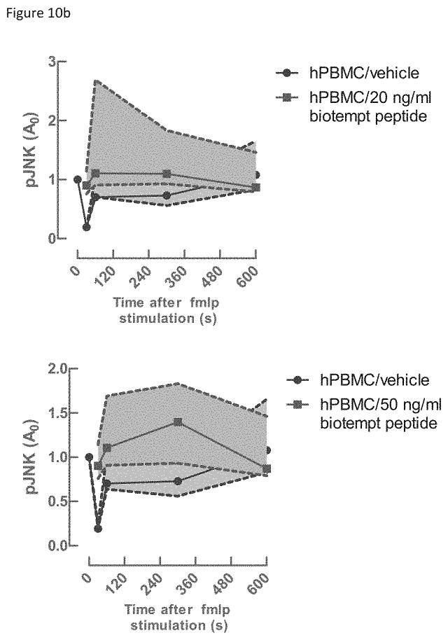

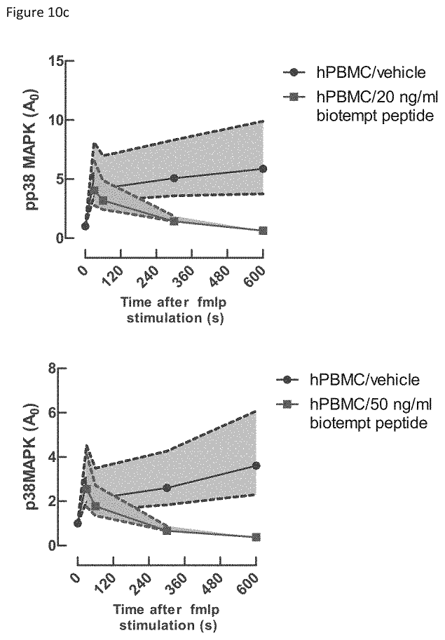

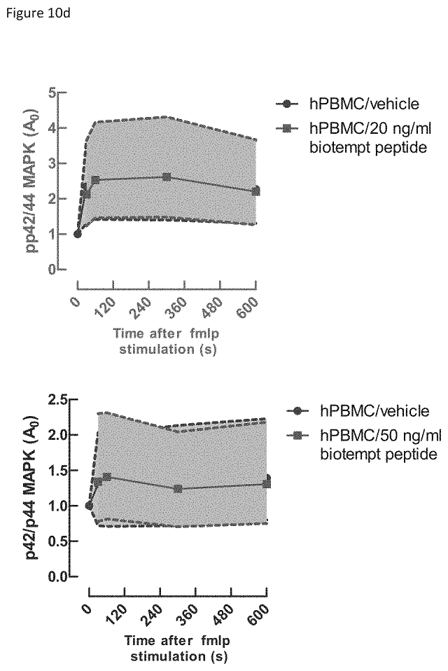

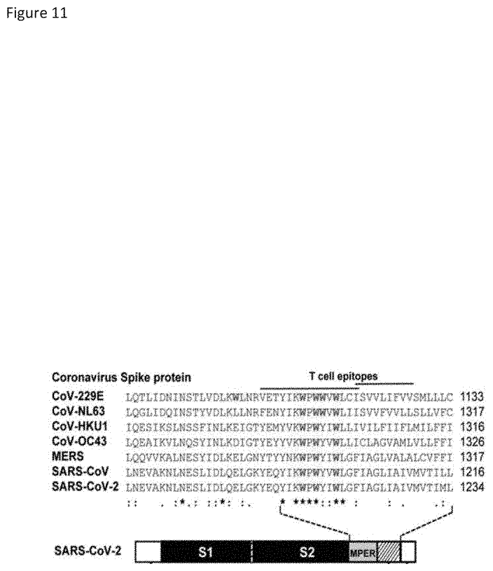

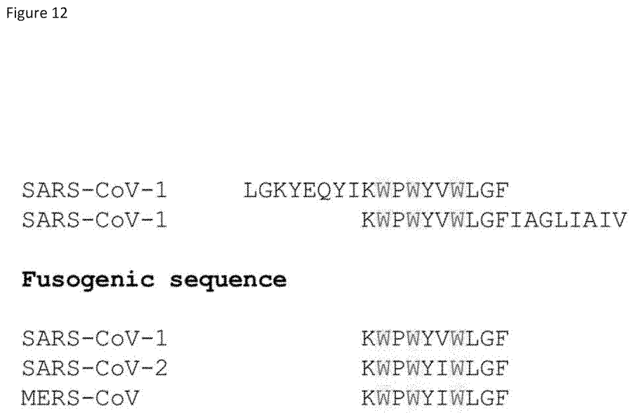

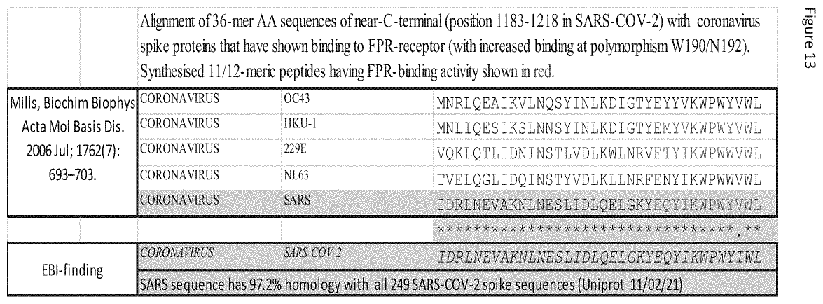

As an immediate serious complication of COVID-19, lung function is severely reduced by accumulation of fluid in the lungs (FIG. 3). This pulmonary edema is caused by increased permeability of blood vessels in the lung as a reaction to the virus infection. Fluid from the vascular network surrounding the alveoli leaks out into the lungs, where this fluid is destroying lung tissue and erasing cells that transport oxygen. The diffuse and profuse increase of adverse fluid and necrotic (dead) cells in both lungs acutely increases the distance that oxygen has to travel through lung and vascular tissues from air to blood, and therewith hampers its exchange from air to all body tissues at large. Vice versa, the diffusion of CO2 from the blood to the air in the lungs is also hampered. These patients typically develop an acute respiratory failure and react with intensely labored breathing, therewith trying to make up for the oxygen shortage they experience.

COVID-19 in the Hospital Cohort.

Roughly one-third of COVID-19 patients with the above two complications (now at around 5-10% of those infected with SARS-Cov-2) have such grave disease that they need to be treated in the hospital. In some countries, most of these patients are admitted to the intensive care unit (ICU). In other countries a smaller group is selected for treatment at the ICU and other patients are either deemed to recover without intensive care treatment or are (treated only palliatively and) left to die. The number of patients admitted to hospital or ICU at one point in time may be immense, due to the steep rise of infection rates seen in a pandemic. As of end-June, 2020, COVID-19 has been confirmed in >8 million people worldwide, now carrying a confirmed case fatality rate of close to 6%. There is an urgent need for effective treatment of this cohort of patients with grave COVID-19.

Ventilator-Associated-Lung-Injury (VALI) and Ventilator-Induced-Lung-Injury (VILI).

Current management of grave COVID-19 is supportive and respiratory failure is the leading cause of mortality (Ruan et al., Intensive Care Med. 2020; DOI:10.1007/s00134-020-05991-x). At the ICU, COVID-19 patients generally are hooked up to a system of mechanical ventilation to provide respiratory relief. However, mechanical ventilation in itself may induce ventilator-assisted-lung injury (VALI; www.ncbi.nlm.nih.gov/pubmed/12559881), and VILI (ventilator-induced-lung-injury with increased edema and aggravated hypoxemia). VALI and VILI (https://www.ncbi.nlm.nih.gov/pubmed/24283226) are recognized herein to play a distinct role in accelerating multiple organ failure associated with COVID-19. Patients requiring mechanical ventilation consume a disproportionately high amount of healthcare resources, both in the ICU and after hospital discharge. Their short-term and long-term mortality is high, and they suffer a very heavy symptom burden for prolonged periods. Hospital survivors have a significant degree of functional and cognitive limitations, and a high readmission rate. Some remain at high risk for death after hospital discharge. Prolonged hospitalization for PMV patients who are at high risk of death does not meet current standards of cost-effectiveness. Consequently, minimizing ventilator requirement and thus minimizing risks on VALI and VILI may paradoxically be key to reduce mortality during COVID-19. Currently no pharmacological methods to combat VALI of VILI are available that address these problems as well as AQGV (SEQ ID NO:2) peptide as provided herein.

AQGV Peptide EA-230 Reduces Adverse Vascular Fluid Permeability.

EA-230 has surprisingly been found to modulate vascular permeability to the good. In particular, EA-230 significantly improves hemodynamic stability after open heart surgery in humans, even in the absence of inflammatory activity of the patient. Permeability governs the amount of fluid leaking from blood vessels. Administration of fluid therapy generally increases leakage. Based on Phase II trial patient observations, a significant reduction of adverse fluid retention (fluid leakage with fluid overload) was found in patients treated with EA-230 (p=0.03). Throughout surgery, EA-230 was shown to be safe and well tolerated. EA-230 given during surgery shows significant improvements in patient recovery after surgery, over placebo patient. EA-230 treated patients are released faster from intensive care (p=0.0232) and hospital (p=0.0015). EA-230 improves hemodynamic stability (p=0.006) and kidney function (p=0.003). Long-term patient recovery was significantly improved by EA-230. By improving vascular permeability, EA-230 can be used to reduce the COVID-19 associated occurrence of adverse fluid in the lungs, and therewith also reduce ventilator use with its detrimental systemic effects.

AQGV (SEQ ID NO:2) Peptide EA-230 Allows Point-of-Care Determination of its Effects on COVID-19 Development.

Moreover, EA-230 has a very short half-life, which facilitates intermittent dosing of the drug and determination of its actual effects at bedside to determine progress of the patient during treatment and make rapid decisions about continuation or discontinuation of treatment. EA230 exhibited a very short elimination half-life and a large volume of distribution (LPS-study: geometric mean and 95% confidence interval: 0.17 [0.12-0.24] hours and 2.2 [1.3-3.8] L/kg, respectively).

Respiratory failure is a common complication not only of COVID-19 and flu but of other respiratory diseases caused by coronaviruses such as SARS and MERS. The phenomenon became more widely known after the 2005 outbreak of the avian H5N1 influenza virus, also known as “bird flu,” when the high fatality rate was linked to an out-of-control systemic multi-organ failure. Now it is a SARS variant, what if next time we have to face a MERS variant? Vaccines and antivirals may differ, fighting respiratory failure stays the same. AQGV peptide can still be used.

Summary of AQGV Peptide EA-230 Effects

Early administration led us detect novel and truly beneficial effects of EA-230 on hemodynamics, kidney function, length of stay in ICU and hospital, that relate to improved hemodynamic stability. Treatment of patients with EA-230 during surgery significantly reduced the need for hemodynamic therapy (combined fluid therapy and blood pressure medication; p=0.006). Besides these improved hemodynamics, EA-230 significantly improved kidney function (as determined by its effects on the glomerular filtration rate) and plasma levels of kidney function biomarker creatinine (p=0.003). It also significantly shortened recovery stay at the ICU and significantly reduced length of stay in the hospital. On average, EA-230-treated patients needed about 8 days of hospital care where placebo-treated patients needed about 10 days. Also, fewer EA-230-treated patients needed re-hospitalization than placebo-treated patients did.

Effects of EA-230 in Human Patients

A prospective, randomized, double-blind, placebo-controlled study was performed in which 180 elective patients undergoing on-pump coronary artery bypass grafting were enrolled. Patients were randomized in a 1:1 ratio and received either EA-230, 90 mg/kg/hour, or a placebo. These were infused at the start of the surgical procedure until the end of the use of the cardiopulmonary bypass. The main focus in this first-in-patient study was on safety and tolerability of EA-230. The primary efficacy endpoint was the modulation of the inflammatory response by EA-230. A key secondary endpoint was the effect of EA-230 on renal function.

Design and Setting

The present study was a single-center, prospective, double-blind, placebo-controlled, randomized, single-dose phase II study. It has an adaptive design to evaluate the safety and immunomodulatory effects of EA-230 in patients undergoing for coronary artery bypass grafting (CABG). 180 eligible patients were included and were randomized to receive either active or placebo treatment in a 1:1 ratio. This was a first-in-patient safety and tolerability study, of which the primary efficacy objective was to assess the immunomodulatory effects of EA-230. The key-secondary efficacy endpoint was the effect of EA-230 on renal function. This study was described in accordance with the Standard Protocol Items: Recommendations for Interventional Trial (SPIRIT) guidelines, and registered at clinicaltrials.gov under number NCT03145220.

Randomization and Stratification

Patients were randomized by non-blinded independent study personnel for active or placebo treatment. Study personnel used Good-Clinical-Practice-approved data management software (Castor EDC, Amsterdam, the Netherlands) in this process. The Castor system applies a stratified randomization to ensure equal distribution between active and placebo treatment of patients with known risk factors for adverse outcomes. Three strata were included: 1) a CABG procedure; 2) pre-operative renal function with an estimated GFR of ≤30, 31-90 and >90 ml/min/1.73 m2; and 3) a EuroSCORE II of <4 or ≥4 (Nashef et al., Eur. J. Cardiothora. Surg. 2012 April; 41(4):734-44).

Blinding

Double-blind conditions were maintained for all patients, the attending physicians and the medical study team personnel involved in all blinded study procedures, data collection and/or data analyses. Non-blinded study personnel not involved in any other study procedures prepared the study medication. Infusion systems and solutions for active and placebo treatment were identical in appearance and texture. Unblinding was authorized by the sponsor after completion of the study, performance of a blinded data review and locking of the database.

Study Intervention

Intravenous infusion of EA-230, 90 mg/kg/hour, or placebo, was initiated at the moment of first surgical incision using an automated infusion pump. Infusion rate was set at 250 mL/hour, and infusion was continued until cessation of the CPB, or after 4 hours of continuous infusion, whichever comes first.

The EA-230 formulation was packed in sterile 5 mL glass vials, containing 1500 mg/vial, dissolved in water for injection at a final concentration of 300 mg/mL with an osmolality of 800 to 1000 mOsm/kg. The placebo formulation consisted of sodium chloride diluted in water for injection in identical sterile 5 mL glass vials containing 29 mg/mL to reach a solution with an identical osmolality. EA-230 and placebo were prepared for continuous intravenous infusion with an osmolality of <400 mOsm/Kg by adding the appropriate amount of EA-230 or placebo to 1000 mL normal saline under aseptic conditions. Placebo and active treatment vials, were manufactured by HALIX BV (Leiden, the Netherlands).

Adverse Events (AEs)

All AEs were judged by the investigators with regard to severity (“mild, moderate, or severe”) according to Common Terminology Criteria for Adverse Events guidelines 4.030 and their perceived relation to the study drug (“definitely, probably, possibly, or unrelated/unlikely to be related”). SAEs or SUSARs include death, life-threatening disease, persistent and/or significant disability and/or incapacity, and hospitalization and/or prolongation of inpatient hospitalization.

Ethical Considerations, Data Quality Assurance & Patient and Public Involvement

The study was conducted in accordance with the ethical principles of the Declaration of Helsinki (ICH E6(R1), the Medical Research Involving Human Subjects Act, guidelines of Good Clinical Practice and European Directive (2001/20/CE). Informed consent was obtained before any study-specific procedures were performed. Data was handled confidentially and anonymously and Good-Clinical-Practice standards were applied. The handling of patient data in this study complies with the Dutch Personal Data Protection Act (in Dutch: Wet Bescherming Persoonsgegevens, WBP). Patients and the public were not involved in the design and/or the conduct of the study protocol. Study outcome was disseminated to all study participants individually. The burden of the intervention was assessed by the independent ethics committees CMO and CCMO, which includes laymen members.

### Results

When assessing the data obtained during the clinical trial, strikingly, no immunomodulatory effects were apparently observed as no significant difference of plasma levels between the EA-230 and placebo group were observed for IL-8, IL-10, IL-1RA, IL-17, MCP-1 and ICAM and other cytokines tested. This was also the case for IL-6 plasma levels, the primary endpoint of the study. Strikingly, significantly less patients suffering from fluid retention were found the EA-230 treatment group (see Table 1). Various parameters were further analyzed and it was found that hemodynamic parameters (such as vasopressor use and/or fluid balance) and/or kidney parameters were advantageously affected by the use of EA-230 as compared with placebo. It was concluded that timing of EA-230 dosing was too early, or at least not sufficiently done during a hyper inflammatory state of the CABG patients.

Surprisingly, however, in the absence of any observed immunomodulatory effects, it was found that the length of stay in the ICU (intensive care unit), and also in hospital, of patients treated with the AQGV (SEQ ID NO:2) peptide, was significantly reduced. Upon an in depth analysis of parameters monitored in the human subjects during the study, it was delineated/found that the use of the AQGV (SEQ ID NO:2) peptide, advantageously modulated the hemodynamics of the treated patients. It was also found that parameters related to kidney function in human patients were shown to have improved significantly, or were maintained and did not deteriorate, even despite the absence of any observed immunomodulatory effects in these patients. Parameters related to kidney function and/or hemodynamics are generally monitored in patients and determine the length of stay in either ICU or hospital. The use of the AQGV (SEQ ID NO:2) thus allows to advantageously improve parameters that are monitored in human patients to thereby reduce the length of stay in either ICU or hospital.

TABLE 1
Adverse events (AEs) in the EASI-study.
AEs, serious adverse events (SAE), and suspected unexpected serious adverse
reaction (SUSAR with differences between treatment groups are listed here. Significantly less (Chi
Square P < 0.05) AEs were found in the EA-230 treatment group (217) than in the placebo treated
group (283). Significantly less patients (Chi Square P < 0.05) suffering from fluid retention were
found the EA-230 treatment group (n = 2) than in the placebo treated group (n = 11), p < 0.05.

| | EA-230 (N = 91) | | | Placebo (N = 89) | | | Overall (N = 180) | | |
|---|---|---|---|---|---|---|---|---|---|
| | e | n | (%) | e | n | (%) | e | n | (%) |
| Any AE | 217 | 78 | (85.7) | 283 | 81 | (91) | 500 | 159 | (88.3) |
| Any SAE | 23 | 12 | (13.2) | 19 | 17 | (19.1) | 32 | 29 | (16.1) |
| Any SUSAR | 0 | 0 | (0) | 1 | 1 | (1.1) | 1 | 1 | (0.6) |
| AE of mild intensity | 188 | 76 | (83.5) | 231 | 78 | (87.6) | 419 | 154 | (85.6) |
| AE of moderate intensity | 23 | 22 | (24.2) | 45 | 27 | (30.3) | 68 | 49 | (27.2) |
| AE of severe intensity | 6 | 5 | (5.5) | 7 | 4 | (4.5) | 13 | 9 | (5) |
| Blood and lymphatic | | | | | | | | | |
| system disorders | | | | | | | | | |
| Overall | 6 | 5 | (5.5) | 8 | 8 | (9) | 14 | 13 | (7.2) |
| Anemia | 5 | 5 | (5.5) | 8 | 8 | (9) | 13 | 13 | (7.2) |
| Hemorrhagic diathesis | 1 | 1 | (1.1) | 0 | 0 | (0) | 1 | 1 | (0.6) |
| Gastrointestinal disorders | | | | | | | | | |
| Overall | 27 | 21 | (23.1) | 36 | 23 | (25.8) | 63 | 44 | (24.4) |
| Nausea | 15 | 15 | (16.5) | 12 | 12 | (13.5) | 27 | 27 | (15) |
| Infections and infestations | | | | | | | | | |
| Overall | 15 | 13 | (14.3) | 22 | 17 | (19.1) | 37 | 30 | (16.7) |
| Metabolism and nutrition | | | | | | | | | |
| disorders | | | | | | | | | |
| Overall | 10 | 10 | (11) | 22 | 14 | (15.7) | 32 | 24 | (13.3) |
| Fluid retention | 2 | 2 | (2.2) | 9 | 9 | (10.1) | 11 | 11 | (6.1) |
| Psychiatric disorders | | | | | | | | | |
| Overall | 9 | 9 | (9.9) | 14 | 13 | (14.6) | 23 | 22 | (12.2) |
| Delirium | 5 | 5 | (5.5) | 9 | 9 | (10.1) | 14 | 14 | (7.8) |
| Renal and urinary | | | | | | | | | |
| disorders | | | | | | | | | |
| Overall | 4 | 4 | (4.4) | 12 | 11 | (12.4) | 16 | 15 | (8.3) |

N = Number of patients involved
n = Number of patients experiencing the event
e = Number of events

TABLE 2
Average on-pump length of patients with average
age of patients, split in quartiles Q1, Q2, Q3 and Q4 of
pump length, and of all patients tested (Q1-Q4).

| | Treatment | Average on-pump-length | Age in years |
|---|---|---|---|
| Quartiles | group | in minutes(+/− SD) | (+/− SD) |
| Q1 | EA-230 | 112 +/− 12 | 68.5 +/− 7.3 |
| Q1 | Placebo | 113 +/− 7 | 70.3 +/− 7.9 |
| Q2 | EA-230 | 137 +/− 5 | 66.5 +/− 9.5 |
| Q2 | Placebo | 136 +/− 6 | 68.1 +/− 6.9 |
| Q3 | EA-230 | 164 +/− 8 | 66.3 +/− 8.7 |
| Q3 | Placebo | 159 +/− 8 | 68.3 +/− 11.0 |
| Q4 | EA-230 | 211 +/− 24 | 65.0 +/− 7.4 |
| Q4 | Placebo | 207 +/− 22 | 64.0 +/− 10.6 |
| Q1-Q4 | EA-230 | 156 +/− 37 | 66.5 +/− 8.3 |
| Q1-Q4 | Placebo | 153 +/− 39 | 67.7 +/− 9.3 |

Hemodynamic Stability in the EASI-Study

In general, the use of vasopressors was reduced in the group that was treated with EA-230. Patients were divided in quartiles based on treatment duration. In Table 3, descriptive frequencies of the 2 variables: days on vasopression and nett fluid balance day 0-2 (first 72 hours) are shown. The groups were split in patients without acute kidney injury (AKI) and with AKI, as well in patients without treatment (placebo) and with treatment with EA-230 (active). EA-230 decreased the net (netto) fluid balance in patients both with and without AKI. EA-230 decreased the need for vasopressors in patients with AKI.

TABLE 3
Frequencies
Statistics

| RIFLE score (incl | | | | days on | Netto fluid |
|---|---|---|---|---|---|
| urine output) | Treatment group | | | vasopression | balance day 0-2 |
| no AKI | Placebo | N | Valid | 42 | 42 |
| | | | Missing | 0 | 0 |
| | | Mean | | 1.38 | 1951.97 |
| | | Median | | 1.00 | 1957.15 |
| | | Std. Deviation | | .795 | 1027.255 |
| | Active | N | Valid | 51 | 51 |
| | | | Missing | 0 | 0 |
| | | Mean | | 1.37 | 1348.47 |
| | | Median | | 1.00 | 1213.70 |
| | | Std. Deviation | | 1.131 | 1304.211 |
| AKI | Placebo | N | Valid | 47 | 47 |
| | | | Missing | 0 | 0 |
| | | Mean | | 2.23 | 3342.04 |
| | | Median | | 2.00 | 2965.20 |
| | | Std. Deviation | | 2.108 | 2275.286 |
| | Active | N | Valid | 39 | 39 |
| | | | Missing | 0 | 0 |

| | | |
|---|---|---|
| Mean | 1.97 | 2549.16 |
| Median | 2.00 | 2328.80 |
| Std. Deviation | 1.423 | 1517.623 |

Modulation of Fluid Balance and Vasopressor Use by Treatment with EA-230

The effects of EA-230 versus placebo were tested in uni- and multivariate models (see table 4). Input/independent variable: treatment group (EA-230 or placebo). Output/dependent variables were: endpoint of fluid balance first 72 hours, days on vasopressin or vasopressor score (area under the curve). Effects of EA-230 versus placebo were tested on two combined variables in model A: fluid balance first 72 hours+days on vasopressin and model B: Fluid balance first 72 hours+vasopressor score AUC. The results of testing in both multivariate models showed significant improvement of hemodynamic parameters in patients receiving EA-230. This was observed in model A (fluid balance first 72 hours+days on vasopressin) p=0.006 and in model B (fluid balance first 72 hours+vasopressor score AUC) p=0.008. In the group of patients that showed no AKI, hemodynamic effects of EA-230 were significantly better as well, illustrating that improvement in hemodynamics can occur independent of kidney failure.

TABLE 4
Goal-directed hemodynamic therapy by EA-230.
An analysis is shown for model A for the total group and for subgroups of acute kidney
injury split conform the RIFLE criteria: No AKI (placebo n = 42, EA-230 n = 50),
Risk (placebo n = 31, EA-230 n = 34), and Injury (placebo n = 16, EA-230 n = 6).
The corresponding p-values are listed.

| | | TREATMENT EFFECTS ON: | | |
|---|---|---|---|---|
| | | Univariate | Univariate | Multivariate |
| Significant improvement of | | 1. Fluid balance | 2. Days on | 1 and 2 |
| active over placebo | | first 72 hours | vasopressin | combined |
| RIFLE stage | Total group | 0.441 | 0.807 | 0.006 |
| | No AKI | 0.017 | 0.996 | 0.048 |
| | Risk | 0.807 | 0.564 | 0.753 |
| | Injury | 0.051 | 0.055 | 0.114 |

Uni- and multivariate general linear model analysis

Combined, these results indicate that the use of EA-230 (SEQ ID NO:2) can improve and/or maintain hemodynamics in human patients, as assessed, inter alia, by affecting the duration of vasopressor use, amount of vasopressor administered and/or fluid balance. In particular, EA-230 improves hemodynamic stability in humans. Permeability governs the amount of fluid leaking from blood vessels. Administration of fluid therapy generally increases leakage. Based on Phase II trial patient observations, a significant reduction of adverse fluid retention (fluid leakage) was found in patients treated with EA-230 (p=0.03). Also, contractility governs tone. It is often adjusted by administration of blood-pressure medications, which, however, may show major detrimental side effects. Based on Phase II trial patient observations, a considerable reduction of required blood pressure medication use was found in the half of patients treated longest with EA-230 (>156 min; p=0.093). It was also determined mean maximum concentrations (mean Cmax) as determined in vivo in humans for EA-230 in the Phase II clinical trial. Mean arterial Cmax found: 30500 ng/mL (range 12500 to 57500 ng/mL). Mean venous Cmax found: 68400 ng/mL (range 19600 to 113000 ng/mL)

EA-230 (SEQ ID NO:2) has an Advantageous Effect on Kidney Function

Effects of EA-230 on modulation in incidence of different stages of acute kidney injury (AKI) were determined according to the RIFLE criteria (RIFLE: risk, injury, failure, loss of kidney function, and end-stage kidney disease classification, Clin. Kidney J. 2013 February; 6(1): 8-14). In the EA-230 group, the number of patients with no AKI increased, whereas the number of patients in the Injury category of the RIFLE criteria decreased. Furthermore, the use of EA-230 significantly improved GFR. Creatinine clearance, a biomarker of kidney function, was significantly improved in patients treated with EA-230. When kidney function was taken into account, clearance of creatinine was significantly improved when EA-230 was used, when kidney function was below 60 mL/minute. When kidney function was above 60 mL/minute, no differences were observed. When pre-treatment kidney function was above 60 mL/minute/1.73 m2, no differences were found between groups. These results indicate that the use of EA-230 can improve and/or maintain kidney function in human patients.

Length of Stay in ICU, Hospital and Readmissions

In the study, effects on length of stay at the ICU of patients and length of stay in the hospital (inpatient care) were investigated. Treatment with EA-230 resulted in a significant reduction of the length of stay (LOS) at the ICU as well as at the hospital. LOS in the ICU and the hospital was reduced in the EA-230 group. The patients treated with EA-230 also showed a considerable (p=0.09) reduction of the number of re-admissions to the hospital up to 90 days after surgery (See Table 5).

TABLE 5
Number of readmissions in the EASI-study (CABG-study). The number of patients that
had to be re-admitted to the hospital due to clinical disease in the period post-treatment.
Readmittance was scored in the period of 28 days after operation, and in the period
ranging from 29-90 days after operation, and for the total period of 90 days after
operation. Readmittance was reduced in patients receiving EA-230 treated group.

| | CABG-study | CABG-study | Total CABG- | |
|---|---|---|---|---|
| Table of | Re-admission | Re-admission | study | Total |
| Re-admissions | day 28 | day 90 | Re-admission | patients |
| Placebo | 5 | 5 | 10 | 89 |
| EA-230 | 2 | 2 | 4 | 91 |
| Total | 7 | 7 | 14 | 180 |

Furthermore, in the patient group treated with AQGV (SEQ ID NO:2), the number of patients suffering from AKI Injury was reduced, and when patients suffered AKI injury, these patients did not have a prolonged length-of-stay, as observed in the placebo group and length of stay was similar to patients having no AKI or patients being at risk of AKI.

Treatment with EA-230 herewith shows strong beneficial effects on recovery. EA-230-treated patients required significantly less hemodynamic therapy, regained post-surgical kidney function significantly faster and remained for a shorter period of time in the Intensive Care Unit (ICU) and in the hospital, as compared to placebo-treated patients.

These novel hemodynamic effects of EA-230 are independent of anti-inflammatory effects of EA-230. In short, significant improvements of hemodynamic stability, kidney function and recovery of EA-230 treated patients relate to novel effects of EA-230 on blood vessel-permeability and blood vessel-contractility. EA-230 shows significant improvements in patient recovery, over placebo patients. EA-230 treated patients are released faster from intensive care (p=0.0232) and hospital (p=0.0015). EA-230 improves hemodynamic stability (p=0.006) and kidney function (p=0.003). While the primary endpoint—short-term inflammatory cytokine (IL-6) reduction—was missed, long-term patient recovery was significantly improved by EA-230.

Significant improvement was found of hemodynamic stability (reducing fluid therapy and blood pressure medication; p=0.006), with: significant improvement of kidney function (improved glomerular filtration rate reduces plasma creatinine; p=0.003), significant reduction of patients suffering from adverse fluid retention during recovery (2 for EA-230, 9 for placebo; p=0.03), and considerable reduction of re-admissions to the hospital in the 90 days after treatment (4 for EA-230, 10 for placebo; p=0.09).

Further Analysis Biomarkers Related to Vasoconstriction and/or Vasodilation.

In view of the effects observed on hemodynamics and lung function, plasma samples are further analyzed with regard to selected biomarkers. Plasma samples of control patients and patients receiving the EA-230 are analyzed with regard to biomarkers Endothelin-1, VEGF, Angiotensin II, ANG2/ANG1 ratio, and cAMP and natriuretic peptides.

In Vitro Effects of EA-230 and AQGV (SEQ ID NO:2) Analogues.

In an in vitro transwell assay the effects of the AQGV peptide (EA-230), and analogues thereof, is tested on human endothelial cells. Briefly, endothelial cells are cultured in transwell culture dishes and culture medium is supplemented with AQGV (SEQ ID NO:2) peptides, and analogues thereof, or control compounds known to affect endothelial layer permeability, vasoconstriction and/or vasodilation. Suitable human endothelial cells are, e.g., HUVECs (Park et al., Stem Cell Rev. 2 (2): 93-102, 2006; Jiménez et al., Cytotechnology 65, 1-14, 2012) and HMEC-1 (Ades E W, et al., J. Invest. Dermatol. 99(6): 683-690, 1992). The permeability of the endothelial layer is determined by measuring the penetration of a macromolecule. Furthermore, levels of biomarkers are also determined in culture medium. Experiments are carried as outlined, e.g., in Cox et al., Shock, 43(4):322-6; 2015. In HUVEC permeability tests, established human endothelial vascular cells (HUVEC), capable of lining blood vessels, are grown in cell-culture (i.e., n=5) on sieves, in multiple test formats, allowing determination of leak-through products depending on various test-concentrations of EA-230 peptide or placebo controls used, establishing pharmacological parameters of EA-230-peptide-effects on permeability in human cells, with or without effectors, such as thrombin, bradykinine, lipopolysaccharide, (LPS), spike protein of coronavirus, nucleic acid of coronavirus, high mobility group box 1 (HMGB1) protein, and reversing their effects with AQGV-peptides.

Also, Bravo et al. (J. Pharmacol. Toxicol. Methods. 2018 January-February; 89:47-53) developed an impedance-based contraction assay using the xCELLigence RTCA MP system. This technology utilizes special 96-well E-plates with gold microelectrode arrays printed in individual wells to monitor cellular adhesion by recording the electrical impedance in real time. The impedance change (percentage vs. control) can be used as the readout for cellular contraction. Established human aortic smooth muscle cells (HaSMC), capable of contracting blood vessels, are grown in cell-culture (i.e., n=3) on gold-electrodes, in multiple test formats, allowing electrical-impedance-determination of endothelin-1 induced smooth muscle cell contractions, depending on various test-concentrations of EA-230-peptide or placebo controls used, establishing pharmacological parameters of EA-230-effects on contractility in human cells. In addition, isolated aneurysmatic (n=3)/control (n=3) patient human aortic smooth muscle cells (APaSMC), differentially capable of contracting blood vessels, are grown in cell-cultures on gold-electrodes in multiple test formats, allowing electrical-impedance-determination of ionomycin-induced smooth muscle cell contractions of patient-versus-control cells, depending on various test-concentrations of EA-230-peptide or placebo controls used, detecting effects of EA-230 in patient cells, with or without effectors, such as thrombin, bradykinin, lipopolysaccharide, (LPS), spike protein of coronavirus, nucleic acid of coronavirus, high mobility group box 1 (HMGB1) protein, and reversing their effects with AQGV-peptides. Similar studies are used to various test-concentrations of EA-230-peptide or placebo controls used, detecting effects of EA-230 in human lung organoid cultures, with or without effectors, such as thrombin, bradykinin, lipopolysaccharide, (LPS), spike protein of coronavirus, nucleic acid of coronavirus, high mobility group box 1 (HMGB1) protein, and reversing their effects with AQGV-peptides. Similar studies are used to various test-concentrations of EA-230-peptide or placebo controls used, detecting effects of EA-230 in experimental mice provided with human ACE2 receptor, with or without effectors, such as thrombin, bradykinin, lipopolysaccharide, (LPS), spike protein of coronavirus, nucleic acid of coronavirus, high mobility group box 1 (HMGB1) protein, and reversing their effects with AQGV-peptides.

Examples of pharmaceutical compositions for use in method of reducing the permeability of an endothelial layer of a blood vessel in a subject, the method comprising:

providing to the endothelial layer a substance that reduces the ratio of Angiopoietin-2 to Angiopoietin-1 at the site of increased permeability as a result of an infection.

### Example 1

| | maleate |
|---|---|
| (SEQ ID NO: 48) | |
| | AQGVLPGQ |
| | To prepare 1L of the composition, |
| | mix maleate -- 1.8 mol 0.9%NaCl -- |
| | 1L |
| (SEQ ID NO: 48) | |
| | AQGVLPGQ |

### Example 2

| | maleate |
|---|---|
| (SEQ ID NO: 49) | |
| | LQGVLPGQ |
| | To prepare 1L of the composition, |
| | mix maleate -- 1.8 mol 0.9%NaCl -- |
| | 1L |
| (SEQ ID NO: 49) | |
| | LQGVLPGQ |

### Example 3

(SEQ ID NO: 50)
AQGLQPGQ-maleate
To prepare 1L of the composition, mix
(SEQ ID NO: 50)
AQGLQPGQ-maleate −1.8 mol
0.9% NaCl-1L

### Example 4

(SEQ ID NO: 51)
LQGLQPGQ-maleate
To prepare 1L of the composition, mix
(SEQ ID NO: 51)
LQGLQPGQ-maleate −1.8 mol
0.9% NaCl-1L

### Example 5

(SEQ ID NO: 52)
AQGV-maleate
To prepare 1L of the composition, mix
(SEQ ID NO: 52)
AQGV-maleate −1.8 mol
0.9% NaCl-1L

### Example 6

(SEQ ID NO: 53)
LQGVL-maleate
To prepare 1L of the composition, mix
(SEQ ID NO: 53)
LQGVL-maleate −1.8 mol
0.9% NaCl-1L

### Example 7

(SEQ ID NO: 54)
AQGLQ-maleate
To prepare 1L of the composition, mix
(SEQ ID NO: 54)
AQGLQ-maleate −1.8 mol
0.9% NaCl-1L

### Example 8

(SEQ ID NO: 55)
LQGLQ-maleate
To prepare 1L of the composition, mix
(SEQ ID NO: 55)
LQGLQ-maleate −1.8 mol
0.9% NaCl-1L

### Example 9

(SEQ ID NO: 56)
AQGVLPGQ-acetate
To prepare 1L of the composition, mix
(SEQ ID NO: 56)
AQGVLPGQ-acetate −1.8 mol
0.9% NaCl-1L

### Example 10

(SEQ ID NO: 57)
LQGVLPGQ-acetate
To prepare 1L of the composition, mix
(SEQ ID NO: 57)
LQGVLPGQ-acetate −1.8 mol
0.9% NaCl-1L

### Example 11

(SEQ ID NO: 58)
AQGLQPGQ-acetate
To prepare 1L of the composition, mix
(SEQ ID NO: 58)
AQGLQPGQ-acetate −1.8 mol
0.9% NaCl-1L

### Example 12

(SEQ ID NO: 59)
LQGLQPGQ-acetate
To prepare 1L of the composition, mix
(SEQ ID NO: 59)
LQGLQPGQ-acetate −1.8 mol
0.9% NaCl-1L

### Example 13

(SEQ ID NO: 60)
AQGV-acetate
To prepare 1L of the composition, mix
(SEQ ID NO: 60)
AQGV-acetate −1.8 mol
0.9% NaCl-1L

### Example 14

(SEQ ID NO: 61)
LQGVL-acetate
To prepare 1L of the composition, mix
(SEQ ID NO: 61)
LQGVL-acetate −1.8 mol
0.9% NaCl-1L

### Example 15

(SEQ ID NO: 62)
AQGLQ-acetate
To prepare 1L of the composition, mix
(SEQ ID NO: 62)
AQGLQPGQ-acetate −1.8 mol
0.9% NaCl-1L

### Example 16

(SEQ ID NO: 63)
LQGLQ-acetate
To prepare 1L of the composition, mix
(SEQ ID NO: 63)
LQGLQ-acetate −1.8 mol
0.9% NaCl-1L

### Example 17

(SEQ ID NO: 64)
AQGVLPGQ-tartrate
To prepare 1L of the composition, mix
(SEQ ID NO: 64)
AQGVLPGQ-tartrate −1.8 mol
0.9% NaCl-1L

### Example 18

(SEQ ID NO: 65)
LQGVLPGQ-tartrate
To prepare 1L of the composition, mix
(SEQ ID NO: 65)
LQGVLPGQ-tartrate −1.8 mol
0.9% NaCl-1L

### Example 19

(SEQ ID NO: 66)
AQGLQPGQ-tartrate
To prepare 1L of the composition, mix
(SEQ ID NO: 66)
AQGLQPGQ-tartrate −1.8 mol
0.9% NaCl-1L

### Example 20

(SEQ ID NO: 67)
LQGLQPGQ-tartrate
To prepare 1L of the composition, mix
(SEQ ID NO: 67)
LQGLQPGQ-tartrate −1.8 mol
0.9% NaCl-1L

### Example 21

(SEQ ID NO: 68)
AQGV-tartrate
To prepare 1L of the composition, mix
(SEQ ID NO: 68)
AQGV-tartrate −1.8 mol
0.9% NaCl-1L

### Example 22

(SEQ ID NO: 69)
LQGVL-tartrate
To prepare 1L of the composition, mix
(SEQ ID NO: 69)
LQGVL-tartrate −1.8 mol
0.9% NaCl-1L

### Example 23

(SEQ ID NO: 70)
AQGLQ-tartrate
To prepare 1L of the composition, mix
(SEQ ID NO: 70)
AQGLQ-tartrate −1.8 mol
0.9% NaCl-1L

### Example 24

(SEQ ID NO: 71)
LQGLQ-tartrate
To prepare 1L of the composition, mix
(SEQ ID NO: 71)
LQGLQ-tartrate −1.8 mol
0.9% NaCl-1L

### Example 25

(SEQ ID NO: 72)
AQGVLPGQ-citrate
To prepare 1L of the composition, mix
(SEQ ID NO: 72)
AQGVLPGQ-citrate −1.8 mol
0.9% NaCl-1L

### Example 26

(SEQ ID NO: 73)
LQGVLPGQ-citrate
To prepare 1L of the composition, mix
(SEQ ID NO: 73)
LQGVLPGQ-citrate −1.8 mol
0.9% NaCl-1L

### Example 27

(SEQ ID NO: 74)
AQGLQPGQ-citrate
To prepare 1L of the composition, mix
(SEQ ID NO: 74)
AQGLQPGQ-citrate −1.8 mol
0.9% NaCl-1L

### Example 28

(SEQ ID NO: 75)
LQGLQPGQ-citrate
To prepare 1L of the composition, mix
(SEQ ID NO: 75)
LQGLQPGQ-citrate −1.8 mol
0.9% NaCl-1L

### Example 29

(SEQ ID NO: 76)
AQGV-citrate
To prepare 1L of the composition, mix
(SEQ ID NO: 76)
AQGV-citrate −1.8 mol
0.9% NaCl-1L

### Example 30

(SEQ ID NO: 77)
LQGVL-citrate
To prepare 1L of the composition, mix
(SEQ ID NO: 77)
LQGVL-citrate −1.8 mol
0.9% NaCl-1L

### Example 31

(SEQ ID NO: 78)
AQGLQ-citrate
To prepare 1L of the composition, mix
(SEQ ID NO: 78)
AQGLQPGQ-citrate −1.8 mol
0.9% NaCl-1L

### Example 32

(SEQ ID NO: 79)
LQGLQ-citrate
To prepare 1L of the composition, mix
(SEQ ID NO: 79)
LQGLQ-citrate −1.8 mol
0.9% NaCl-1L

1. A method for reducing the permeability of an endothelial layer of a blood vessel in a subject, the method comprising:
providing to the endothelial layer a substance that reduces the ratio of Angiopoietin-2 to Angiopoietin-1 at the site of increased permeability as a result of an infection.

2. The method according to claim 1, wherein the subject is a human suffering from a respiratory infection, and wherein the method reduces the gas diffusion distance between lung-alveoli and vascular network surrounding alveoli in the subject so as to treat the respiratory infection.

3. The method according to claim 1, wherein the substance comprises an AQGV peptide (SEQ ID NO:2), an LQGV peptide (SEQ ID NO:3), or a functional analogue of either.

4. The method according to claim 1, wherein the infection is a viral infection.

5. The method according to claim 4, wherein the viral infection is caused by a virus requiring a specific receptor and a more ubiquitous binding partner present on at least a percentage of lung alveolar cells.

6. The method according to claim 5, wherein the specific receptor is ACE-2.

7. The method according to claim 4, wherein the more ubiquitous binding partner is a glycoprotein comprising a sialic acid residue.

8. The method according to claim 4, wherein the virus is a coronavirus.

9. A method of treating a human subject suffering from an infection of COVID-19 or a mutant of COVID-19, the method comprising:
providing to the endothelial layer of a blood vessel of the human subject at a site of increased permeability due to the infection, a substance that reduces the ratio of angiopoietin-2 to angiopoietin-1
so as to reduce the increased permeability of the endothelial layer of the blood vessel due to the infection.

10. The method according to claim 1, wherein the substance is administered intravenously to the subject.

11. The method according to claim 1, wherein the substance is administered intermittently to the subject.

12. The method according to claim 1, further comprising monitoring the subject for hemodynamic stability.

13. The method according to claim 1, further comprising administering an antiviral agent to the subject.

14. (canceled)

15. (canceled)

16. The method according to claim 10, wherein the substance is administered intravenously at a rate of at least 75 mg/kg/hour.

17. The method according to claim 16, wherein the substance is administered intravenously at a rate of at least 90 mg/kg/hour.

18. The method according to claim 9, wherein the substance comprises the peptide of SEQ ID NO:2, the peptide of SEQ ID NO:3, a functional analogue of SEQ ID NO:2, or a functional analogue of SEQ ID NO:3.

19. The method according to claim 18, wherein the substance is administered intravenously to the human subject.

20. The method according to claim 19, wherein the substance is administered intravenously at a rate of at least 75 mg/kg/hour.

21. The method according to claim 20, wherein the substance is administered intravenously at a rate of at least 90 mg/kg/hour.

22. The method according to claim 1, further comprising monitoring the subject for hemodynamic stability.
